(19) United States
(12) Patent Application Publication
(10) Pub. No.: US 2022/0162327 A1
(43) Pub. Date: May 26, 2022

(54) ANTI-CCR5 AGENTS AND METHODS OF TREATMENT THAT BLOCK CANCER METASTASIS OR ENHANCE CELL DEATH INDUCED BY DNA DAMAGING CHEMOTHERAPY

(71) Applicant: CytoDyn Inc., Vancouver, WA (US)

(72) Inventors: Scott KELLY, Vancouver, WA (US); Richard G. Pestell, Vancouver, WA (US)

(21) Appl. No.: 17/598,650

(22) PCT Filed: Apr. 1, 2020
(86) PCT No.: PCT/US2020/026253

§ 371 Date: Sep. 27, 2021

Related U.S. Application Data
(60) Provisional application No. 62/827,729, filed on Apr. 1, 2019.

(51) Int. Cl. C07K 16/28; A61K 31/704; A61P 35/00

(52) U.S. Cl. CPC C07K 16/2866; A61K 2039/545; A61P 35/00; A61K 31/704

(57) ABSTRACT

The present disclosure relates to the use of DNA damaging agents and leronlimab (PRO 140), or other anti-CCR5 agents, to treat or prevent cancer metastases and enhance the cell killing ability of the DNA damaging agents by selectively targeting the CCR5 receptor. The present disclosure relates to the use of DNA damaging agents and leronlimab (PRO 140), or other anti-CCR5 agents, to treat or prevent cancer metastases and reduce circulating tumor cells (CTC) or putative metastatic tumor cells in the peripheral blood following treatment, reduce CCR5 expression on cancer-associated cells after following treatment, decrease volume in tumor size following treatment. The present disclosure may be used to treat or prevent subjects with cancer and, particularly, subjects with metastatic CCR5+ cancer.

### BACKGROUND

For cancer, research has shown that CCR5 plays an important role in tumor invasion and metastasis. Increased CCR5 expression is an indicator of disease status in several cancers. And published studies have shown that blocking CCR5 can reduce tumor metastases in laboratory and animal models of aggressive breast and prostate cancer.

Some studies have indicated that CCR5 signaling has anti-tumor effects, acting as a co-stimulatory molecule for T cell activation and increasing T cell chemotaxis to the tumor microenvironment. See Gao et al., CCL5 activation of CCR5 regulates cell metabolism to enhance proliferation of breast cancer cells, OPEN BIOL., 6: 160122 (2016); Gonzalez-Martin et al., CCR5 in cancer immunotherapy: More than an “attractive” receptor for T cells, ONCOIMMUNOLOGY, 1: 106-108 (2012). However, evidence also suggests that CCL5/CCR5 axis signaling may be preferentially activated in certain types of cancers, for example breast and prostate cancers, and that such signaling facilitates disease progression. For example, some studies indicate that cancer cells can overexpress CCL5, CCR5, or both, likely contributing to their growth and proliferation via the effects of CCR5 signaling on mechanistic target of rapamycin (mTOR). See Gao et al., CCL5 activation of CCR5 regulates cell metabolism to enhance proliferation of breast cancer cells, OPEN BIOL., 6: 160122 (2016); see also Chow and Luster, Chemokines in Cancer, CANCER IMMUNOL. RES., 2(12): 1125-1131 (2014); Singh et al., Expression of CCR5 and its ligand CCL5 in pancreatic cancer (Abstract), J IMMUNOL, 196(1 Supplement): 51.3 (2016). Additionally, some immunosuppressive immune cells, including regulatory T cells (Treg) and myeloid-derived suppressor cells (MDSC), express CCR5, suggesting another pathway by which CCR5 signaling may contribute to tumor growth. Mukaida, CCR5 antagonist, an ally to fight against metastatic colorectal cancer, TRANSLATIONAL CANCER RESEARCH, 5(Supp. 2): S309-S312 (2016). Furthermore, it has been reported that cancer cells in the tumor microenvironment can exploit CCL5 production by CD4⁺ and CD8⁺ T cells to lead to increased tumor growth and tumor cell spreading. Halama et al., Tumoral Immune Cell Exploitation in Colorectal Cancer Metastases Can Be Targeted Effectively by Anti-CCR5 Therapy in Cancer Patients, CANCER CELL, 29: 587-601 (2016).

Exploratory efforts using anti-CCR5 binding agents to alter CCL5/CCR5 signaling in connection with some cancer types have been made. Sicoli et al., CCR5 Receptor Antagonists Block Metastasis to Bone ofv-Src Oncogene-Transformed Metastatic Prostate Cancer Cell Lines, CANCER RES., 74(23): 7103-7114 (2014); Velasco-Velázquez et al., The CCL5/CCR5 Axis Promotes Metastasis In Basal Breast Cancer, ONCOIMMUNOLOGY, 2(4): e23660 (2013); Velasco-Velázquez et al., CCR5 Antagonist Blocks Metastasis of Basal Breast Cancer Cells, CANCER RES., 72(15): 3839-3850 (2012). Various compounds exist that inhibit, interrupt, block, alter, or modify the CCR5/CCL5 receptor/ligand axis (i.e., CCR5 receptor/CCL5 ligand axis). Many of these compounds have been developed for the treatment of HIV-1, which also binds with the CCR5 receptor and is known to share some binding commonalities with CCL5. Such compounds include extracellular or cell transmembrane CCR5 binding agents such as, for example, PRO 140 (extracellular) and maraviroc (transmembrane), and other compounds such as vicriviroc, aplaviroc, SCH-C, and TAK-779, and antibodies such as PA14, 2D7, RoAb13, RoAb14, 45523, etc. It has been found that the most potently antiviral anti-CCR5 monoclonal antibodies including, for example, PRO 140, bind CCR5 receptor amino acid residues in EL2 alone or in combination with Nt residues. It has also been determined that the CCR5 receptor binding sites for anti-CCR5 monoclonal antibodies are distinct from those of small-molecule CCR5 antagonists. That is, available small-molecule CCR5 antagonists, such as maraviroc, bind the hydrophobic cavity formed by the transmembrane helices, i.e., not the extracellular Nt or loop regions. The amino acid residue E283 in the seventh transmembrane region has been specifically identified as a principle site or interaction for small molecules, and maraviroc and vicriviroc have been found to bind to identical sets of CCR5 receptor amino acids. Olson et al., CCR5 Monoclonal Antibodies for HIV-1 Therapy, CURR. OPIN. HIV AIDS, March, 4(2): 104-111 (2009). It has also been reported, however, that the CCL5 ligand and maraviroc dock on the CCR5 receptor by sharing two receptor sites: the Nt and the ECL2, and that synthetic CCL5-derived peptides may also be used to block the CCR5 receptor. Secchi et al., Combination of the CCL5-Derived Peptide R4.0 with Different HIV-1 Blockers Reveals Wide Target Compatibility and Synergic Cobinding to CCR5, ANTIMICROB AGENTS CHEMOTHER., 58(10): 6215-6223 (2014).

In some instances, CCL5 expression associated with immune cell activation can be exploited by cancer cells in the tumor microenvironment, and blocking CCR5 signaling using inhibitors such as maraviroc may have anti-tumor effects. In a study of human colorectal cancer liver metastasis, CD4⁺ and CD8⁺ T cells at the invasive margin expressed CCL5, which was associated with T cell exhaustion, tumor proliferation, invasive tumor cell behavior, and increased production of matrix metalloproteinases by tumor-associated macrophages. Halama et al., Tumoral Immune Cell Exploitation in Colorectal Cancer Metastases Can Be Targeted Effectively by Anti-CCR5 Therapy in Cancer Patients, CANCER CELL, 29: 587-601 (2016). Inhibiting CCL5 with maraviroc led to repolarization of tumor-associated macrophages and tumor cell death. Halama et al. (2016).

However, inhibition of CCR5 signaling can also have immunosuppressive effects. In vitro studies have been conducted to investigate the effects of CCR5 receptor blockade by maraviroc on activated human T cells on potential immunological mechanisms. It was found that blocking CCR5 by maraviroc not only can block CCR5 and CCR2 internalization processes induced by CCL5 and CCL2, but can also inhibit T cell chemotactic activities toward their cognate ligands, respectively. Further, blocking CCR5 with maraviroc at high doses tends to decrease production of TNF-α and IFN-γ. It was also noted that the effect of maraviroc on CCR5 was temporary and reversible. Yuan et al., In Vitro Immunological Effects of Blocking CCR5 on T Cells, INFLAMMATION, 38(2): 902-910 (2015); see Arberas et al., In vitro effects of the CCR5 inhibitor maraviroc on human T cell function, J. ANTIMICROB. CHEMOTHER., 68(3): 577-586 (2013).

In view of the numerous and sometimes contradictory roles of CCR5 signaling in contributing to tumor development, there exists a need for competitive inhibitors to the CCR5 receptor and methods of use that can be used to inhibit, dampen, interrupt, block, alter, or modify the CCR5/CCL5 receptor/ligand axis for therapeutic purposes without triggering, or that reduce the impact of, unintended side effects. Further, there is a need for such competitive inhibitors to the CCR5 receptor and methods of use that cause fewer and less severe side effects, are longer-lasting, and facilitate improved patient compliance due to decreased dosing demands and improved patient experience (due to fewer undesirable side effects), including side effects caused by the competitive inhibitor itself. Optimal therapeutic modalities using the CCL5/CCR5 axis as a therapeutic target will need to accommodate two opposing demands: the need to inhibit the detrimental involvement of CCL5 and CCR5 in specific malignant diseases while protecting their potentially beneficial activities in immunity.

Breast cancer continues to be the most common solid tumor affecting women, and it is the second leading cause of cancer-related death in women. Metastasis is the primary cause of death in patients with breast cancer. Currently no approved treatments exist that are directed specifically to the metastatic process.

Also, ten to fifteen percent of breast cancer patients have Triple Negative Breast Cancer (TNBC), which is defined by the lack of estrogen receptor (ER), progesterone receptor (PgR) and human epidermal growth factor receptor-2 (HER-2) expression, which are known targets of endocrine therapies and anti-HER2 agents, respectively. Approximately 70-84% of TNBCs are basal-like; conversely, about 70% of basal-like tumors are TNBCs (Nielson 2004, Prat 2011, Prat 2013).

Chemotherapy is still the main treatment option for TNBC patients, and standard treatment is surgery with adjuvant chemotherapy and radiotherapy. Although TNBC responds to chemotherapeutic agents such as taxanes and anthracyclines better than other subtypes of breast cancer, prognosis still remains poor. As a variation, neoadjuvant chemotherapy is frequently used for triple-negative breast cancers [Hudis 2011]. This allows for a higher rate of breast-conserving surgeries and, from evaluating the response to the chemotherapy, gives important clues about the individual responsiveness of the particular cancer to chemotherapy.

Due to the loss of target receptors such as ER, PGR, and HER-2, patients with TNBC do not benefit from hormonal or trastuzumab-based therapy. Hence, surgery and chemotherapy, individually or in combination, appear to be the only available modalities. To date there are multiple approaches attempting to improve care of triple negative breast cancer patients, including DNA damaging agents like platinum, targeted EGFR and VEGF inhibitors, and, PARP inhibitors; however, none have been as clinically successful as anticipated and more targeted therapies need to be developed and explored [Aysola 2013]. Thus, metastatic TNBC is a complex disease with an unmet need and an unproven treatment regimen in clinics.

Improved cancer treatments directed specifically to the metastatic process, such as administration of a CCR5 binding agent together with currently available therapies such as, for example, a DNA damaging agent, are needed provide meaningful improvements to patient treatment options.

### BRIEF SUMMARY

The present disclosure relates to the use of DNA damaging agents and leronlimab (PRO 140), or other anti-CCR5 agents, to treat or prevent cancer metastases and enhance the cell killing ability of the DNA damaging agents by selectively targeting the CCR5 receptor. The present disclosure relates to the use of DNA damaging agents and leronlimab (PRO 140), or other anti-CCR5 agents, to treat or prevent cancer metastases and reduce circulating tumor cells (CTC) or putative metastatic tumor cells in the peripheral blood following treatment, reduce CCR5 expression on cancer-associated cells after following treatment, decrease volume in tumor size following treatment. The present disclosure may be used to treat or prevent subjects with cancer and, particularly, subjects with metastatic CCR5+ cancer.

As shown in the xenograft models described herein, leronlimab can effectively block CCR5 positive breast cancer metastasis. Also provided are murine xenograft models that show that, by reducing the ability of breast cancer cells to metastasize, tumors are more contained. Additionally, it is shown that leronlimab can potentially provide standard DNA damaging chemotherapies more time to work, potentially providing significantly improved efficacy of existing cancer therapies with fewer side effects. That is, leronlimab enhances the effect of DNA damaging agents to kill cancer cells.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

FIGS. 1A and 1B. Leronlimab binds CCR5 in human breast cancer cells.

FIGS. 2A-2D. PRO140 blocks human CCR5-mediated signaling in human breast cancer cells.

FIGS. 3A-3D. Leronlimab blocks CCR5-mediated invasion of human breast cancer cells into extracellular matrix.

FIGS. 4A and 4B. Leronlimab block breast cancer metastasis in mice.

FIGS. 5A and 5B. Leronlimab enhances the cell death induced by Doxorubicin, a DNA damage inducing chemotherapy agent.

FIGS. 6A-6C. FIGS. 6A-6C show immunohistochemical staining for CCR5 in tissue samples from a subject with triple negative breast cancer. FIG. 6A shows representative images of IHC for CCR5. Immunohistochemistry analysis on archival tissue showed high predominance of CCR5+ tumor infiltrating leukocytes.

### DETAILED DESCRIPTION

Increased CCR5 expression is an indicator of disease status in several cancers including, but not limited to, breast cancer.

Although metastasis is the leading cause of death for patients with breast cancer, currently there are no treatments available that are directed to the metastatic process. Thus, better treatments for metastatic cancer, including metastatic breast cancer are needed. Presented herein are methods for treating a subject for metastatic breast cancer by administering to the subject an effective amount of a CCR5 binding agent, such as leronlimab. In particular, the present disclosure relates to the use of leronlimab (PRO 140), or other anti-CCR5 agents, to treat, reduce, prevent, or block cancer metastases and/or enhance the cell killing ability of DNA damaging chemotherapy by selectively targeting the CCR5 receptor in subjects with cancer.

Preclinical and clinical data have suggested that chemokine receptors and its ligands, also referred as chemoattractant or chemotactic cytokines, are involved in the process of cancer cells tropism by specific organs [Moser, 2001][Neagu, 2015][Velasco-Velazquez, 2012]. C-C Chemokine receptor type-5 (CCR5) is selectively reexpressed on the surface of tumor cells during the dedifferentiation and transformation process (velasco-velazquez-2012). Velasco-Velazquez et al. have evaluated an analysis of a combined microarray database comprising 2,254 breast cancer samples and showed that expression of CCL5/CCR5 is higher in basal subtypes (over 58% of samples) of breast cancer compared to luminal subtypes [Velasco-Velazquez, 2012]. CCR5 has been shown to be sufficient to induce in vitro invasiveness and metastasis of breast cancer cells that is blocked by CCR5 inhibitors [Velasco-Velazquez, 2012]. CCR5 inhibitors, such as Maraviroc, effectively blocked lung metastases in breast cancer tumor model.

CCR5 binding agents, including leronlimab (PRO 140), show a significant reduction in tumor volume in a breast cancer tumor model. Another cancer hallmark that CCR5 presents a potential role is the DNA repair pathways. This cancer characteristic attenuates apoptosis and contributes to chemotherapy resistance and tumor cells immortality. Studies have correlated the altered expression of C-C Chemokine Ligand type-5 (CCL5) with disease progression in patients with breast cancer [Luboshits, 1999] [Niwa, 2001] [Zhang, 2009].

CCR5 binding agents, such as antagonists Maraviroc and Vicriviroc, dramatically enhanced cell killing mediated by DNA-damaging chemotherapeutic agents. Single-cell analysis revealed CCR5 governs PI3K/Akt, ribosomal biogenesis, and cell survival signaling [Jiao-2018].

The role of CCR5 blockade of the CCL5-CCR5 pathway in immune control of tumors has recently been shown and provided new horizon to target this deadly disease [de Oliveira, 2017, Del Prete, 2017, Lanitis, 2017]. CCR5 immunohistochemistry of biopsies allows to selectively choosing patients with CCR5 expression not only on tumor but on intra-tumor immune cells in the tumor microenvironment.

Targeted therapy with one or more CCR5 binding agents, such as leronlimab (PRO 140), may have a potential to increase overall response rate due to a synergy in DNA crosslink strand break of chemotherapeutic agents, such as carboplatin, and reduce DNA repair secondary to CCR5 binding by Leronlimab (PRO 140).

### Glossary

Prior to setting forth this disclosure in more detail, it may be helpful to an understanding thereof to provide definitions of certain terms to be used herein. Unless defined otherwise, all technical and scientific terms used herein have the same meaning as is commonly understood by one of skill in the art to which this invention belongs. Additional definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as dose, are to be understood to include any integer within the recited range, unless otherwise indicated. As used herein, the term “about” means±20% of the indicated range, value, or structure, unless otherwise indicated.

It should be understood that the terms “a” and “an” as used herein refer to “one or more” of the enumerated components. The use of the alternative (e.g., “or”) should be understood to mean either one, both, or any combination thereof of the alternatives.

As used herein, the terms “include,” “have,” and “comprise” are used synonymously, which terms and variants thereof are intended to be construed as non-limiting.

The term “consisting essentially of” limits the scope of a claim to the specified materials or steps, or to those that do not materially affect the basic characteristics of a claimed invention. For example, a protein domain, region, or module (e.g., a binding domain, hinge region, linker module) or a protein (which may have one or more domains, regions, or modules) “consists essentially of” a particular amino acid sequence when the amino acid sequence of a domain, region, or module or protein includes extensions, deletions, mutations, or any combination thereof (e.g., amino acids at the amino- or carboxy-terminus or between domains) that, in combination, contribute to at most 20% (e.g., at most 15%, 10%, 8%, 6%, 5%, 4%, 3%, 2%, or 1%) of the length of a domain, region, or module or protein and do not substantially affect (i.e., do not reduce the activity by more than 50%, such as no more than 40%, 30%, 25%, 20%, 15%, 10%, 5%, or 1%) the activity of the domain(s), region(s), module(s), or protein (e.g., the target binding affinity of a binding protein).

As used herein, “chemokine” means a cytokine that can stimulate leukocyte movement. Chemokines may be characterized as either cys-cys or cys-X-cys depending on whether the two amino terminal cysteine residues are immediately adjacent or separated by one amino acid. It includes, but is not limited to, CCL5 (also known as RANTES), MIP-1α, MIP-1β, or SDF-1, or another chemokine which has similar activity.

As used herein, “chemokine receptor” means a member of a homologous family of seven-transmembrane spanning cell surface proteins that bind chemokines.

As used herein, “CCR5” is a chemokine receptor which binds members of the C-C group of chemokines and whose amino acid sequence comprises that provided in Genbank Accession Number 1705896, and related polymorphic variants. As used herein, “antibody” means an immunoglobulin molecule comprising two heavy chains and two light chains and that recognizes an antigen. The immunoglobulin molecule may derive from any of the commonly known classes or isotypes, including but not limited to IgA, secretory IgA, IgG, and IgM. IgG subclasses are also well known to those in the art and include but are not limited to human IgG1, IgG2, IgG3, and IgG4. It includes, by way of example, both naturally occurring and non-naturally occurring antibodies. Specifically, “antibody” includes polyclonal and monoclonal antibodies, and monovalent and divalent fragments thereof. Furthermore, “antibody” includes chimeric antibodies, wholly synthetic antibodies, single chain antibodies, and fragments thereof. Optionally, an antibody can be labeled with a detectable marker. Detectable markers include, for example, radioactive or fluorescent markers. The antibody may be a human or nonhuman antibody. The nonhuman antibody may be humanized by recombinant methods to reduce its immunogenicity in humans. Methods for humanizing antibodies are known to those skilled in the art.

As used herein, “monoclonal antibody,” also designated as “mAb,” is used to describe antibody molecules whose primary sequences are essentially identical and which exhibit the same antigenic specificity. Monoclonal antibodies may be produced by hybridoma, recombinant, transgenic, or other techniques known to one skilled in the art.

As used herein, “heavy chain” means the larger polypeptide of an antibody molecule composed of one variable domain (VH) and three or four constant domains (CH1, CH2, CH3, and CH4), or fragments thereof.

As used herein, “light chain” means the smaller polypeptide of an antibody molecule composed of one variable domain (VL) and one constant domain (CL), or fragments thereof. As used herein, a “binding fragment” or an “antigen-binding fragment or portion” of an antibody refers to the fragment or portion of an intact antibody that has or retains the ability to bind to the antigen target molecule recognized by the intact antibody, including fragment antigen binding (Fab) fragments, F(ab′)2 fragments, Fab′ fragments, Fv fragments, recombinant IgG (rIgG) fragments, single chain antibody fragments, including single chain variable fragments (scFv), and single domain antibodies (e.g., sdAb, sdFv, nanobody) fragments. The term encompasses genetically engineered or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, tetrabodies, tandem di-scFv, and tandem tri-scFv.

As used herein, “Fab” means a monovalent antigen binding fragment of an immunoglobulin that consists of one light chain and part of a heavy chain. It can be obtained by brief papain digestion or by recombinant methods.

As used herein, “F(ab′)2 fragment” means a bivalent antigen binding fragment of an immunoglobulin that consists of both light chains and part of both heavy chains. It can be obtained by brief pepsin digestion or recombinant methods.

As used herein, “CDR” or “complementarity determining region” means a highly variable sequence of amino acids in the variable domain of an antibody.

As used herein, “humanized” describes antibodies wherein some, most or all of the amino acids outside the CDR regions are replaced with corresponding amino acids derived from human immunoglobulin molecules. In one embodiment of the humanized forms of the antibodies, some, most, or all of the amino acids outside the CDR regions have been replaced with amino acids from human immunoglobulin molecules but where some, most, or all amino acids within one or more CDR regions are unchanged. Small additions, deletions, insertions, substitutions, or modifications of amino acids are permissible as long as they would not abrogate the ability of the antibody to bind a given antigen. Suitable human immunoglobulin molecules would include IgG1, IgG2, IgG3, IgG4, IgA, and IgM molecules. A “humanized” antibody would retain a similar antigenic specificity as the original antibody, e.g., in the present disclosure, the ability to bind CCR5.

One skilled in the art would know how to make the humanized antibodies of the subject disclosure. Various publications, several of which are hereby incorporated by reference into this application, also describe how to make humanized antibodies. For example, the methods described in U.S. Pat. No. 4,816,567 comprise the production of chimeric antibodies having a variable region of one antibody and a constant region of another antibody. U.S. Pat. No. 5,225,539 describes another approach for the production of a humanized antibody. This patent describes the use of recombinant DNA technology to produce a humanized antibody wherein the CDRs of a variable region of one immunoglobulin are replaced with the CDRs from an immunoglobulin with a different specificity such that the humanized antibody would recognize the desired target but would not be recognized in a significant way by the human subject's immune system. Specifically, site directed mutagenesis is used to graft the CDRs onto the framework.

Other approaches for humanizing an antibody are described in U.S. Pat. Nos. 5,585,089 and 5,693,761 and WO 90/07861, which describe methods for producing humanized immunoglobulins. These have one or more CDRs and possible additional amino acids from a donor immunoglobulin and a framework region from an accepting human immunoglobulin. These patents describe a method to increase the affinity of an antibody for the desired antigen. Some amino acids in the framework are chosen to be the same as the amino acids at those positions in the donor rather than in the acceptor. Specifically, these patents describe the preparation of a humanized antibody that binds to a receptor by combining the CDRs of a mouse monoclonal antibody with human immunoglobulin framework and constant regions. Human framework regions can be chosen to maximize homology with the mouse sequence. A computer model can be used to identify amino acids in the framework region which are likely to interact with the CDRs or the specific antigen and then mouse amino acids can be used at these positions to create the humanized antibody.

The above U.S. Pat. Nos. 5,585,089 and 5,693,761 and WO 90/07861 also propose four possible criteria which may be used in designing the humanized antibodies. The first proposal was that for an acceptor, use a framework from a particular human immunoglobulin that is unusually homologous to the donor immunoglobulin to be humanized, or use a consensus framework from many human antibodies. The second proposal was that if an amino acid in the framework of the human immunoglobulin is unusual and the donor amino acid at that position is typical for human sequences, then the donor amino acid rather than the acceptor may be selected. The third proposal was that in the positions immediately adjacent to the 3 CDRs in the humanized immunoglobulin chain, the donor amino acid rather than the acceptor amino acid may be selected. The fourth proposal was to use the donor amino acid residue at the framework positions at which the amino acid is predicted to have a side chain atom within 3A of the CDRs in a three dimensional model of the antibody and is predicted to be capable of interacting with the CDRs. The above methods are merely illustrative of some of the methods that one skilled in the art could employ to make humanized antibodies. The affinity and/or specificity of the binding of the humanized antibody may be increased using methods of directed evolution as described in Wu et al., J. MOL. BIOL., 284:151 (1999) and U.S. Pat. Nos. 6,165,793; 6,365,408; and 6,413,774.

The variable regions of the humanized antibody may be linked to at least a portion of an immunoglobulin constant region of a human immunoglobulin. In one embodiment, the humanized antibody contains both light chain and heavy chain constant regions. The heavy chain constant region usually includes CH1, hinge, CH2, CH3, and, sometimes, CH4 region. In one embodiment, the constant regions of the humanized antibody are of the human IgG4 isotype.

The antibodies, or binding fragments, disclosed herein may either be labeled or unlabeled. Unlabeled antibodies can be used in combination with other labeled antibodies (second antibodies) that are reactive with a humanized antibody, such as antibodies specific for human immunoglobulin constant regions. Alternatively, the antibodies can be directly labeled. A wide variety of labels can be employed, such as radionuclides, fluors, enzymes, enzyme substrates, enzyme cofactors, enzyme inhibitors, ligands (particularly haptens), etc. Numerous types of immunoassays are available and are well known to those skilled in the art for detection of CCR5-expressing cells or detection of CCR5 modulation on cells capable of expressing CCR5.

The present disclosure also provides antibody or antibody fragment-polymer conjugates having an effective size or molecular weight, or incorporate other half-life extension technologies, that confer an increase in serum half-life, an increase in mean residence time in circulation (MRT), and/or a decrease in serum clearance rate over underivatized antibody fragments. Antibody fragment-polymer conjugates can be made by derivatizing the desired antibody fragment with an inert polymer. It will be appreciated that any inert polymer which provides the conjugate with the desired apparent size or which has the selected actual molecular weight is suitable for use in constructing antibody fragment-polymer conjugates of the disclosure.

Many inert polymers are suitable for use in pharmaceuticals. See, e.g., Davis et al., Biomedical Polymers: Polymeric Materials and Pharmaceuticals for Biomedical Use, pp. 441-451 (1980). For the antibody or antibody fragment-polymer conjugates disclosed herein, a non-proteinaceous polymer is used. The nonproteinaceous polymer ordinarily is a hydrophilic synthetic polymer, i.e., a polymer not otherwise found in nature. However, polymers which exist in nature and are produced by recombinant or in vitro methods are also useful, as are polymers which are isolated from native sources. Hydrophilic polyvinyl polymers fall within the scope of this disclosure, e.g., polyvinyl alcohol and polyvinylpyrrolidone. Particularly useful are polyalkylene ethers such as polyethylene glycol (PEG); polyoxyalklyenes such as polyoxyethylene, polyoxypropylene, and block copolymers of polyoxyethylene and polyoxypropylene (Pluronics); polymethacrylates; carbomers; branched or unbranched polysaccharides which comprise the saccharide monomers D-mannose, D- and L-galactose, fucose, fructose, D-xylose, L-arabinose, D-glucuronic acid, sialic acid, D-galacturonic acid, D-mannuronic acid (e.g., polymannuronic acid, or alginic acid), D-glucosamine, D-galactosamine, D-glucose, and neuraminic acid including homopolysaccharides and heteropolysaccharides such as lactose, amylopectin, starch, hydroxyethyl starch, amylose, dextran sulfate, dextran, dextrins, glycogen, or the polysaccharide subunit of acid mucopolysaccharides, e.g., hyaluronic acid, polymers of sugar alcohols such as polysorbitol and polymannitol, heparin, or heparon. The polymer prior to cross-linking need not be, but preferably is, water soluble but the final conjugate must be water soluble. Preferably, the conjugate exhibits a water solubility of at least about 0.01 mg/ml and more preferably at least about 0.1 mg/ml, and still more preferably at least about 1 mg/ml. In one embodiment, the polymer should not be highly immunogenic in the conjugate form, nor should it possess viscosity that is incompatible with intraveneous infusion or injection if the conjugate is intended to be administered by such routes.

In one embodiment, the polymer contains only a single group which is reactive. This helps to avoid cross-linking of protein molecules. However it is within the scope of the disclosure to maximize reaction conditions to reduce cross-linking, or to purify the reaction products through gel filtration or ion-exchange chromatography to recover substantially homogeneous derivatives. In other embodiments, the polymer contains two or more reactive groups for the purpose of linking multiple antibody fragments to the polymer backbone.

Gel filtration or ion-exchange chromatography can be used to recover the desired derivative in substantially homogeneous form.

The molecular weight of the polymer can range up to about 500,000 D and preferably is at least about 20,000 D, or at least about 30,000 D, or at least about 40,000 D. The molecular weight chosen can depend upon the effective size of the conjugate to be achieved, the nature (e.g., structure such as linear or branched) of the polymer and the degree of derivitization, i.e., the number of polymer molecules per antibody fragment, and the polymer attachment site or sites on the antibody fragment.

The polymer can be covalently linked to the antibody fragment through a multifunctional crosslinking agent which reacts with the polymer and one or more amino acid residues of the antibody fragment to be linked. However, it is also within the scope of the disclosure to directly crosslink the polymer by reacting a derivatized polymer with the antibody fragment, or vice versa.

The covalent crosslinking site on the antibody fragment includes the N-terminal amino group and epsilon amino groups found on lysine residues, as well other amino, imino, carboxyl, sulfhydryl, hydroxyl, or other hydrophilic groups. The polymer may be covalently bonded directly to the antibody fragment without the use of a multifunctional (ordinarily bifunctional) crosslinking agent, as described in U.S. Pat. No. 6,458,355.

The degree of substitution with such a polymer will vary depending upon the number of reactive sites on the antibody fragment, the molecular weight, hydrophilicity and other characteristics of the polymer, and the particular antibody fragment derivitization sites chosen. In general, the conjugate contains from 1 to about 10 polymer molecules, but greater numbers of polymer molecules attached to the antibody fragments of the disclosure are also contemplated. The desired amount of derivitization is easily achieved by using an experimental matrix in which the time, temperature, and other reaction conditions are varied to change the degree of substitution, after which the level of polymer substitution of the conjugates is determined by size exclusion chromatography or other means known in the art.

Functionalized PEG polymers to modify the antibody fragments of the disclosure are available from Shearwater Polymers, Inc. (Huntsville, Ala.). Such commercially available PEG derivatives include, but are not limited to, amino-PEG, PEG amino acid esters, PEG-hydrazide, PEG-thiol, PEG-succinate, carboxymethylated PEG, PEG-propionic acid, PEG amino acids, PEG succinimidyl succinate, PEG succinimidyl propionate, succinimidyl ester of carboxymethylated PEG, succinimidyl carbonate of PEG, succinimidyl esters of amino acid PEGs, PEG-oxycarbonylimidazole, PEG-nitrophenyl carbonate, PEG tresylate, PEG-glycidyl ether, PEG-aldehyde, PEG-vinylsulfone, PEG-maleimide, PEG-orthopyridyl-disulfide, heterofunctional PEGs, PEG vinyl derivatives, PEG silanes, and PEG phospholides. The reaction conditions for coupling these PEG derivatives will vary depending on the protein, the desired degree of PEGylation, and the PEG derivative utilized. Some factors involved in the choice of PEG derivatives include: the desired point of attachment (such as lysine or cysteine R-groups), hydrolytic stability and reactivity of the derivatives, stability, toxicity and antigenicity of the linkage, suitability for analysis, etc. Specific instructions for the use of any particular derivative are available from the manufacturer. The conjugates of which may be separated from the unreacted starting materials by gel filtration or ion exchange HPLC.

As used herein, “anti-chemokine receptor antibody” means an antibody which recognizes and binds to an epitope on a chemokine receptor. As used herein, “anti-CCR5 antibody” means a monoclonal antibody that recognizes and binds to an epitope on the CCR5 chemokine receptor.

As used herein, “epitope” means a portion of a molecule or molecules that forms a surface for binding antibodies or other compounds. The epitope may comprise contiguous or noncontiguous amino acids, carbohydrate, or other nonpeptidyl moieties or oligomer-specific surfaces.

As used herein, “polypeptide” means two or more amino acids linked by a peptide bond.

A “nucleic acid molecule,” or “polynucleotide,” may be in the form of RNA or DNA, which includes cDNA, genomic DNA, and synthetic DNA. A nucleic acid molecule may be double stranded or single stranded, and if single stranded, may be the coding strand or non-coding (anti-sense strand). A coding molecule may have a coding sequence identical to a coding sequence known in the art or may have a different coding sequence, which, as the result of the redundancy or degeneracy of the genetic code, or by splicing, can encode the same polypeptide.

“Analogs” of antibodies or binding fragments include molecules differing from the antibodies or binding fragments by conservative amino acid substitutions. For purposes of classifying amino acid substitutions as conservative or non-conservative, amino acids may be grouped as follows: Group I (hydrophobic side chains): met, ala, val, leu, ile; Group II (neutral hydrophilic side chains): cys, ser, thr; Group III (acidic side chains): asp, glu; Group IV (basic side chains): asn, gln, his, lys, arg; Group V (residues influencing chain orientation): gly, pro; and Group VI (aromatic side chains): trp, tyr, phe. Conservative substitutions involve substitutions between amino acids in the same class. Non-conservative substitutions constitute exchanging a member of one of these classes for a member of another.

Due to the degeneracy of the genetic code, a variety of nucleic acid sequences encode the proteins or polypeptides disclosed herein. For example, homologous nucleic acid molecules may comprise a nucleotide sequence that is at least about 90% identical to a reference nucleotide sequence. More preferably, the nucleotide sequence is at least about 95% identical, at least about 97% identical, at least about 98% identical, or at least about 99% identical to a reference nucleotide sequence. The homology can be calculated using various, publicly available software tools well known to one of ordinary skill in the art. Exemplary tools include the BLAST system available from the website of the National Center for Biotechnology Information (NCBI) at the National Institutes of Health.

One method of identifying highly homologous nucleotide sequences is via nucleic acid hybridization. Thus, homologous nucleic acid molecules hybridize under high stringency conditions. Identification of related sequences can also be achieved using polymerase chain reaction (PCR) and other amplification techniques suitable for cloning related nucleic acid sequences. Preferably, PCR primers are selected to amplify portions of a nucleic acid sequence of interest, such as a CDR.

The term “high stringency conditions” as used herein refers to parameters with which the art is familiar. Nucleic acid hybridization parameters may be found in references that compile such methods, e.g., MOLECULAR CLONING: A LABORATORY MANUAL, J. Sambrook, et al., eds., Second Edition, Cold Spring Harbor Laboratory Press, Cold Spring Harbor, N.Y., (1989), or CURRENT PROTOCOLS IN MOLECULAR BIOLOGY, F. M. Ausubel, et al., eds., John Wiley & Sons, Inc., New York. One example of high stringency conditions is hybridization at 65 degrees Centigrade in hybridization buffer (3.5×SSC, 0.02% Ficoll, 0.02% polyvinyl pyrrolidone, 0.02% Bovine Serum Albumin, 2.5 mM NaH2PO4 (pII7), 0.5% SDS, 2 mM EDTA). SSC is 0.15M sodium chloride/0.015M sodium citrate, pH7; SDS is sodium dodecyl sulphate; and EDTA is ethylenediaminetetracetic acid. After hybridization, a membrane upon which the nucleic acid is transferred is washed, for example, in 2×SSC at room temperature and then at 0.1-0.5×SSC/0.1×SDS at temperatures up to 68 degrees Centigrade.

As used herein, the term “vector” refers to a nucleic acid molecule that is capable of transporting another nucleic acid. Vectors may be, for example, plasmids, cosmids, viruses, or phage. An “expression vector” is a vector that is capable of directing the expression of a protein encoded by one or more genes carried by the vector when it is present in the appropriate environment.

Nucleic acid sequences may be expressed in hosts after the sequences have been operably linked to (i.e., positioned to ensure the functioning of) an expression control sequence. These expression vectors are typically replicable in the host organisms, either as episomes or as an integral part of the host chromosomal DNA. Commonly, expression vectors will contain selection markers, e.g., tetracycline or neomycin, to permit detection of those cells transformed with the desired DNA sequences. See, e.g., U.S. Pat. No. 4,704,362, which is incorporated herein by reference.

Once expressed, the whole antibodies, their dimers, individual light and heavy chains, or other immunoglobulin forms or binding fragments of the present disclosure, can be purified according to standard procedures of the art, including ammonium sulfate precipitation, affinity columns, column chromatography, gel electrophoresis, and the like. See generally, R. Scopes, PROTEIN PURIFICATION, Springer-Verlag, New York (1982). Substantially pure immunoglobulins of at least about 90 to 95% homogeneity are preferred, and 98 to 99% or more homogeneity most preferred, for pharmaceutical uses. Once purified, partially or to homogeneity as desired, the polypeptides may then be used therapeutically (including extracorporeally) or in developing and performing assay procedures, immunofluorescent stainings, and the like. See generally, IMMUNOLOGICAL METHODS, Vols. I and II, Lefkovits and Pernis, eds., Academic Press, New York, N.Y. (1979 and 1981).

As used herein, “inhibits” means that the amount is reduced in the presence of a composition as compared with the amount that would occur without the composition.

The term “competitive inhibitor” as used herein refers to a molecule that competes with a reference molecule for binding to a target, and thereby blunts, inhibits, dampens, reduces, or blocks the effects of the reference molecule on the target. For example, PRO 140 is a competitive inhibitor of CCL5 binding to CCR5 receptor.

“Agonist activity” as used in the present disclosure refers to the binding by a molecule to a target, wherein the binding activates the target to produce a response.

“CCL5 agonist activity,” as used herein, refers to activity consistent with activation by CCL5.

“Antagonist activity” as used in the present disclosure refers to the binding by a molecule to a target, wherein the binding does not activate the target to produce a response and the binding blocks the action of one or more agonist molecules.

As used herein, “subject” means any animal or artificially modified animal capable of having cancer. Artificially modified animals include, but are not limited to, SCID mice with human immune systems. The animals include but are not limited to mice, rats, dogs, guinea pigs, ferrets, rabbits, and primates. In a preferred embodiment, the subject is a human.

As used herein, “treating” means slowing, stopping, or reversing the progression of a given disease or disorder. In a preferred embodiment, “treating” means reversing the progression of the disease or disorder. In some embodiments, treating includes reversing the progression of the disease or disorder to the point of eliminating the disease or disorder.

As used herein, “preventing” refers to preventing a disease or disorder from occurring; delaying the progression of a disease or disorder; or reducing the pathology or symptomatology of a disease or disorder. For example, preventing a cancer includes preventing the development of a tumor, slowing the growth of a tumor, and delaying the development of a tumor.

As used herein, “administering” may be effected or performed using any of the methods known to one skilled in the art. The methods may comprise oral, intravenous, intramuscular, or subcutaneous means.

As used herein, “effective dose” means an amount in sufficient quantities to either treat the subject or prevent the subject from developing cancer. A person of ordinary skill in the art can perform simple titration experiments to determine what amount is required to treat the subject.

### CCR5 Binding Agent

In one aspect, the present disclosure relates to the use of CCR5 binding agents that target CCR5 receptor, and act as competitive inhibitors to the CCR5 cell receptor without providing CCL5 agonist activity in addition to DNA damaging agents.

In one embodiment, the present disclosure provides for the use of a PRO 140 antibody, or binding fragment thereof, in treating or preventing cancer. PRO 140 is a humanized monoclonal antibody described in U.S. Pat. Nos. 7,122,185 and 8,821,877, which are incorporated herein by reference, in their entirety. PRO 140 is a humanized version of the murine mAb, PA14, which was generated against CD4+ CCR5+ cells. Olson et al., Differential Inhibition of Human Immunodeficiency Virus Type 1 Fusion, gp 120 Binding and CC-Chemokine Activity of Monoclonal Antibodies to CCR5, J. VIROL., 73: 4145-4155. (1999). PRO 140 binds to CCR5 expressed on the surface of a cell, and potently inhibits HIV-1 entry and replication at concentrations that do not affect CCR5 chemokine receptor activity in vitro and in the hu-PBL-SCID mouse model of HIV-1 infection. Olson et al., Differential Inhibition of Human Immunodeficiency Virus Type 1 Fusion, gp 120 Binding and CC-Chemokine Activity of Monoclonal Antibodies to CCR5, J. VIROL., 73: 4145-4155. (1999); Trkola et al., Potent, Broad-Spectrum Inhibition of Human Immunodeficiency Virus Type 1 by the CCR5 Monoclonal Antibody PRO 140, J. VIROL., 75: 579-588 (2001).

Nucleic acids encoding heavy and light chains of the humanized PRO 140 antibody have been deposited with the ATCC. Specifically, the plasmids designated pVKHuPRO140, pVg4-HuPRO140 (mut B+D+I) and pVg4-HuPRO140 HG2, respectively, were deposited pursuant to, and in satisfaction of, the requirements of the Budapest Treaty with the ATCC, Manassas, Va., U.S.A. 20108, on Feb. 22, 2002, under ATCC Accession Nos. PTA 4097, PTA 4099, and PTA 4098, respectively. The American Type Culture Collection (ATCC) is now located at 10801 University Boulevard, Manassas, Va. 20110-2209.

In a one embodiment, the methods disclosed herein comprise administering a humanized antibody designated PRO 140 or an antibody that competes with PRO 140 for binding to the CCR5 receptor, wherein the PRO 140 comprises (i) two light chains, each light chain comprising the expression product of the plasmid designated pVK:HuPRO140-VK (ATCC Deposit Designation PTA-4097), and (ii) two heavy chains, each heavy chain comprising the expression product of either the plasmid designated pVg4:HuPRO140 HG2-VH (ATCC Deposit Designation PTA-4098) or the plasmid designated pVg4:HuPRO140 (mut B+D+I)-VH (ATCC Deposit Designation PTA-4099). In a further embodiment, the PRO 140 is a humanized or human antibody that binds to the same epitope as that to which antibody PRO 140 binds. In another embodiment, the monoclonal antibody is the humanized antibody designated PRO 140.

In a further embodiment, the present disclosure relates to the use of the human antibody designated CCR5mAb004, or a binding fragment thereof. CCR5mAb004 is a fully human mAb, generated using the Abgenix XenoMouse® technology, that specifically recognizes and binds to CCR5. See Roschke et al., Characterization of a Panel of Novel Human Monoclonal Antibodies That Specifically Antagonize CCR5 and Block HIV Entry, 44th Annual Interscience CONFERENCE ON ANTIMICROBIAL AGENTS AND CHEMOTHERAPY, Washington, D.C., Oct. 30-Nov. 2, 2004 (2004); HGS Press Release, Human Genome Sciences Characterizes Panel of Novel Human Monoclonal Antibodies That Specifically Antagonize the CCR5 Receptor and Block HIV-1 Entry, Nov. 2, 2004 (2004); HGS Press Release, Human Genome Sciences Begins Dosing of Patients in a Phase 1 Clinical Trial of CCR5 mAb in Patients Infected With HIV-1, Mar. 30, 2005 (2005).

In one embodiment, the present disclosure relates to the use of the monoclonal antibody PA14, produced by the hybridoma cell line designated PA14 (ATCC Accession No. HB-12610), a binding fragment thereof, or an antibody that competes with monoclonal antibody PA-14 in binding to the CCR5 receptor, in treating or preventing cancer.

In one embodiment, a CCR5 binding agent may comprise a small molecule such as, for example, vicriviroc, UK-427,857, maraviroc, GW873140, TAK-652, Takeda AMD070, or the like.

In one embodiment of the methods described herein, the antibody or binding fragment thereof comprises a light chain of the antibody. In another embodiment, the antibody or binding fragment thereof comprises a heavy chain of the antibody. In a further embodiment, the antibody or binding fragment thereof comprises an Fab portion of the antibody. In a still further embodiment, the antibody or binding fragment thereof comprises an F(ab′)2 portion of the antibody. In an additional embodiment, the antibody or binding fragment thereof comprises an Fd portion of the antibody. In another embodiment, the antibody or binding fragment thereof comprises an Fv portion of the antibody. In a further embodiment, the antibody or binding fragment thereof comprises a variable domain of the antibody. In a still further embodiment, the antibody or binding fragment thereof comprises one or more CDR domains of the antibody. In yet another embodiment, the antibody or binding fragment thereof comprises six CDR domains of the antibody.

The CC-chemokine receptor CCR5 is the major co-receptor for macrophage-tropic (R5) strains, and plays a crucial role in the sexual transmission of HIV-1. It has been demonstrated that tyrosines and negatively charged residues in the amino-terminal domain (Nt) of CCR5 are essential for gp120 binding to the co-receptor, and for HIV-1 fusion and entry. Residues in the extracellular loops (ECL) 1-3 of CCR5 were dispensable for co-receptor function, yet the CCR5 inter-domain configuration had to be maintained for optimal viral fusion and entry (24). This led to the conclusion either that gp120 forms interactions with a diffuse surface on the ECLs, or that the Nt is maintained in a functional conformation by bonds with residues in the ECLs. Studies with chimeric co-receptors and anti-CCR5 monoclonal antibodies have also shown the importance of the extracellular loops for viral entry.

The G protein coupled receptor CCR5, is normally expressed on a subset of T cells and serves as a co-receptor for HIV infection. CCR5 is a requisite fusion co-receptor for primary HIV-1 isolates. PRO140 is an anti-CCR5 monoclonal antibody that potently inhibits HIV-1 entry and replication at concentrations that do not affect CCR5's chemokine receptor activity in vitro. During malignant transformation CCR5 expression is known to increase in a number of cancers (breast cancer (BCa), prostate cancer, colon cancer, melanoma). CCR5 targeted cancer clinical trials using small molecular inhibitors opened to accrual in late 2018. CCR5 is expressed in >50% of human BCa, primarily in triple negative BCa. Its expression in human BCa correlates with poor outcome and CCR5⁺ BCa epithelial cells have characteristics of cancer stem cells, forming mammospheres and initiating tumors with >60-fold greater efficiency in mice. Reintroduction of CCR5 expression into CCR5 negative BCa cells promotes tumor metastases and induces DNA repair gene expression and activity. The CCR5 inhibitor leronlimab has been used for treatment of >660 patients with HIV, including meeting its primary endpoints in a phase III study, without significant adverse events reported.

Here, it is reported that leronlimab bound to CCR5 expressed in human breast cancer cell lines with 98% efficiency. Leronlimab abrogated CCL5 induced Ca⁺² flux and blocked 3D matrigel invasion of MDA-MB-231 cells. CCL5 (C-C chemokine ligand 5), an inflammatory chemokine also known as regulated upon activation and normal T cell expressed and secreted (RANTES), plays an important role in these immunologic mechanisms. CCL5 acts as a key regulator of T cell migration to inflammatory sites, directing migration of T cells to damaged or infected sites. CCL5 also regulates T cell differentiation. Many biologic effects of chemokines are mediated by their interaction with chemokine receptors on cell surfaces. In the present disclosure, the most relevant known receptor for CCL5 is the CCR5 receptor; however, CCR1 and CCR3 are also known CCL5 receptors and CCR4 and CD44 are auxiliary receptors.

The CCR5 receptor is a C-C chemokine G-coupled protein receptor expressed on lymphocytes (e.g., NK cells, B cells), monocytes, macrophages, dendritic cells, a subset of T cells, etc. The CCR5 receptor spans the cellular plasma membrane seven times in a serpentine manner. The extracellular portions represent potential targets for antibodies targeting CCR5, and comprise an amino-terminal domain (Nt) and three extracellular loops (ECL1, ECL2, and ECL3). The extracellular portions of CCR5 comprise just 90 amino acids distributed over four domains. The largest of these domains are at the Nt and ECL2 at approximately 30 amino acids each.

The formation of the CCL5 ligand and CCR5 receptor complex causes a conformational change in the receptor that activates the subunits of the G-protein, inducing signaling and leading to changed levels of cyclic AMP (cAMP), inositol triphosphate, intracellular calcium, and tyrosine kinase activation. These signaling events cause cell polarization and translocation of the transcription factor NF-kB, which results in the increase of phagocytic ability, cell survival, and transcription of proinflammatory genes. Once G-protein dependent signaling occurs, the CCL5/CCR5 receptor complex is internalized via endocytosis.

A complete complex structure of CCL5 in complex with CCR5 has been computationally derived. It is reported that the 1-15 residue moiety of CCL5 is inserted into the CCR5 binding pocket; the 1-6 N-terminal domain of CCL5 is buried within the transmembrane region of CCR5; and the 7-15 residue moiety of CCL5 is predominantly encompassed by the N-terminal domain and extracellular loops of CCR5. CCL5 residues Ala16 and Arg17 and additional residues of the 24-50 residue moiety interact with the upper N-terminal domain and extracellular loop interface of CCR5. It is further reported that the integrity of the amino terminus of CCL5 is crucial to receptor binding and cellular activation. Further, it has been reported that CCL5 and HIV-1 primarily interact with mostly the same CCR5 residues, and share the same chemokine receptor binding pocket.

CCR5 signaling has anti-tumor effects, acting as a co-stimulatory molecule for T cell activation and increasing T cell chemotaxis to the tumor microenvironment. The CCL5/CCR5 axis signaling may be preferentially activated in certain types of cancers, for example breast and prostate cancers, and that such signaling facilitates disease progression. Cancer cells may overexpress CCL5, CCR5, or both, likely contributing to their growth and proliferation via the effects of CCR5 signaling on mechanistic target of rapamycin (mTOR). Additionally, some immunosuppressive immune cells, including regulatory T cells (Treg) and myeloid-derived suppressor cells (MDSC), express CCR5, suggesting another pathway by which CCR5 signaling may contribute to tumor growth. Cancer cells in the tumor microenvironment can exploit CCL5 production by CD4⁺ and CD8⁺ T cells to lead to increased tumor growth and tumor cell spreading.

PRO 140 (Leronlimab) binds with CCR5 receptor and is known to share some binding commonalities with CCL5. Leronlimab binds CCR5 receptor amino acid residues in EL2 alone or in combination with Nt residues. This binding to the CCR5 receptor binding sites for anti-CCR5 monoclonal antibodies is distinct from those of small-molecule CCR5 binding agents.

It has previously been shown that the monoclonal antibody PRO 140 does not affect cAMP levels when added to CD4+ T cells alone, but diminishes the effect of CCL5 on cAMP levels when administered with CCL5. Similarly, although PRO 140 alone does not affect chemotaxis of CHO-K1 cells, PRO 140 reduces CCL5-induced chemotaxis when administered with CCL5. PRO 140 does not have agonist activity for CCR5 but acts as a competitive inhibitor with CCL5 for binding to CCR5.

Leronlimab blocks human breast cancer xenograft metastasis in mice. Leronlimab also augmented cell killing by DNA damage inducing agents including Doxorubicin.

It has been found that leronlimab binds CCR5 in BCa cells, blocking breast cancer cellular invasion and tumor metastasis, and augmenting cell killing by DNA damage inducing chemotherapies. As CCR5 augments DNA repair and is expressed selectively on cancerous but not normal breast epithelial cells, leronlimab may enhance the tumor specific activities of DNA damage response (DDR)-based treatments, allowing a reduction in dose of chemotherapy and radiation.

The studies described herein assess the binding and functional interaction of the humanized monoclonal antibody to CCR5 (Leronlimab) with human breast cancer cell lines.

### Methods of Use

In one aspect, the present disclosure provides methods of treating or preventing a cancer comprising administering to a subject in need thereof a competitive inhibitor to a CCR5 cell receptor. In a particular embodiment, a method for preventing a cancer is provided.

In one embodiment, the present disclosure provides a method of preventing a cancer comprising administering to a subject in need thereof a competitive inhibitor to a CCR5 cell receptor that does not itself have CCL5 agonist activity is provided, wherein the competitive inhibitor binds to the ECL-2 loop of the CCR5 cell receptor. In a further embodiment, the competitive inhibitor competes with CCL5 for binding to the CCR5 cell receptor. In a further embodiment, the competitive inhibitor comprises the monoclonal antibody PRO 140, or a binding fragment thereof. In a further embodiment, the competitive inhibitor competes for binding with the monoclonal antibody PRO 140, or a binding fragment thereof.

In one embodiment, the present disclosure provides a method of preventing a cancer comprising administering to a subject in need thereof: (a) a PRO 140 antibody, or binding fragment thereof; (b) a nucleic acid encoding a PRO 140 antibody, or binding fragment thereof; (c) a vector comprising a nucleic acid encoding a PRO 140 antibody, or binding fragment thereof; or (d) a host cell comprising (i) a PRO 140 antibody, or binding fragment thereof, (ii) a nucleic acid encoding a PRO 140 antibody, or binding fragment thereof, or (iii) a vector comprising a nucleic acid encoding a PRO 140 antibody, or binding fragment thereof. In the aforementioned embodiment, the PRO 140 antibody, or binding fragment thereof, may comprise, for example, a PRO 140 monoclonal antibody or a scFv.

In one embodiment, the present disclosure provides a method of preventing a cancer comprising administering to a subject in need thereof a PRO 140 antibody, or binding fragment thereof.

In one embodiment, the competitive inhibitor to a CCR5 cell receptor, such as PRO 140, is administered with a pharmaceutically acceptable carrier. Pharmaceutically acceptable carriers are well known to those skilled in the art. Such pharmaceutically acceptable carriers may include but are not limited to aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, saline, and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, chelating agents, inert gases, and the like.

The dose of the composition of the invention will vary depending on the subject and upon the particular route of administration used. Dosages can range from 0.1 to 100,000 μg/kg. Based upon the composition, the dose can be delivered continuously, such as by continuous pump, or at periodic intervals, e.g., on one or more separate occasions. Desired time intervals of multiple doses of a particular composition can be determined without undue experimentation by one skilled in the art.

In one embodiment of the instant methods, the antibody or binding fragment thereof is administered to the subject a plurality of times and each administration delivers from 0.01 mg per kg body weight to 50 mg per kg body weight of the antibody or binding fragment thereof to the subject. In another embodiment, each administration delivers from 0.05 mg per kg body weight to 25 mg per kg body weight of the antibody or binding fragment thereof to the subject. In a further embodiment, each administration delivers from 0.1 mg per kg body weight to 10 mg per kg body weight of the antibody or binding fragment thereof to the subject. In a still further embodiment, each administration delivers from 0.5 mg per kg body weight to 5 mg per kg body weight of the antibody or binding fragment thereof to the subject. In another embodiment, each administration delivers from 1 mg per kg body weight to 3 mg per kg body weight of the antibody or binding fragment thereof to the subject. In another embodiment, each administration delivers about 2 mg per kg body weight of the antibody or binding fragment thereof to the subject.

In one embodiment, the antibody or binding fragment thereof is administered a plurality of times, and a first administration is separated from the subsequent administration by an interval of less than one week. In another embodiment, the first administration is separated from the subsequent administration by an interval of at least one week. In a further embodiment, the first administration is separated from the subsequent administration by an interval of one week. In another embodiment, the first administration is separated from the subsequent administration by an interval of two to four weeks. In another embodiment, the first administration is separated from the subsequent administration by an interval of two weeks. In a further embodiment, the first administration is separated from the subsequent administration by an interval of four weeks. In yet another embodiment, the antibody or binding fragment thereof is administered a plurality of times, and a first administration is separated from the subsequent administration by an interval of at least one month.

In a further embodiment, the antibody or binding fragment thereof is administered to the subject via intravenous infusion. In another embodiment, the antibody or binding fragment thereof is administered to the subject via subcutaneous injection. In another embodiment, the antibody or binding fragment thereof is administered to the subject via intramuscular injection.

In some embodiments, the PRO 140 is administered at a once weekly dose of 350 mg to 1400 mg, or about 525 mg or about 700 mg or about 1050 mg. In some embodiments, the PRO 140 is administered at a twice weekly dose of 350 mg to 1400 mg, or about 525 mg or about 700 mg or about 1050 mg.

In some embodiments, PRO 140 is administered in a formulation comprising concentrated PRO 140 in an amount greater than about 100 mg/mL and less than about 200 mg/mL; a tonicifier consisting essentially of a sodium salt and a histidine and glycine buffer present in a combined amount of from about 110 mM to about 120 mM and wherein the buffer is present in an amount of about 10 mM to about 25 mM; and a surfactant, wherein the formulation is hypotonic and has a total salt concentration of less than 100 mM.

In some embodiments, PRO 140 is administered in a formulation comprising: concentrated PRO 140 in an amount greater than about 100 mg/mL and less than about 200 mg/mL; a sodium salt in an amount greater than about 90 mM and less than 100 mM; a histidine and glycine buffer in an amount greater than about 5 mM and less than about 25 mM; a surfactant in an amount greater than about 0.001% w/v and less than about 0.2% w/v; and, optionally, a stabilizing agent or non-salt tonicifier in an amount of about 0.05% w/v to about 1.8% w/v; wherein the formulation has an osmolality of about 250 to about 280 mOsm and has a total salt concentration of less than 100 mM.

In some embodiments, PRO 140 is administered in a low viscosity, hypotonic formulation, comprising: (a) concentrated PRO 140 in an amount greater than about 100 mg/mL and less than about 200 mg/mL; (b) a sodium salt in an amount selected from about 90 mM or about 95 mM; (c) a histidine and glycine buffer in an amount of about 20 mM; (d) a surfactant in an amount of 0.005% to 0.2% w/v; and optionally (e) a stabilizing agent or non-salt tonicifier in an amount sufficient to provide an osmolality of the formulation of about 260-280 mOs/kg; wherein the formulation has a total salt concentration of less than 100 mM.

In some embodiments, PRO 140 is administered in a low viscosity hypotonic formulation, comprising: (a) concentrated PRO 140 in an amount greater than about 100 mg/mL and less than about 200 mg/mL; (b) a salt in an amount selected from about 90 mM or about 95 mM, wherein the salt is selected from sodium chloride, sodium gluconate, or sodium lactate; (c) a histidine and glycine buffer in an amount of about 20 mM; (d) a surfactant in an amount of about 0.005% to about 0.2% w/v, wherein the surfactant is a polysorbate, a poloxamer, or a pluronic; and (e) a stabilizing agent or non-salt tonicifier present in an amount sufficient to provide an osmolality of the formulation of about 230 mOs/kg to about 280 mOs/kg, wherein the stabilizing agent or non-salt tonicifier is selected from a sugar alcohol, a monosaccharide, a disaccharide, or a combination thereof; wherein the formulation has a total salt concentration of less than 100 mM.

In some embodiments, PRO 140 is administered in a composition comprising PRO 140 in an amount greater than about 100 mg/mL and less than about 200 mg/mL, a tonicifier comprising a sodium salt present in a concentration of greater than about 90 mM and a histidine and glycine buffer present in a combined amount of from 110 mM to 120 mM and a surfactant present in an amount of from about 0.001% to about 0.2% w/v, wherein the composition has an osmolality of about 230 to about 290 mOs/kg and a total salt concentration of less than 100 mM.

In some embodiments, PRO 140 is provided as an article of manufacture comprising a container and a formulation comprising PRO 140 in a concentration of greater than 100 mg/mL and less than 200 mg/mL, a tonicifier of a sodium salt present in a concentration of greater than about 90 mM and a histidine and glycine buffer present in a combined amount of from about 110 mM to about 120 mM and the formulation has a total salt concentration of less than 100 mM, a surfactant in an amount of from about 0.005% to about 0.2%, and instructions for use.

In some embodiments, PRO 140 will be administered in a dose of 700 mg of Leronlimab (PRO 140) (175 mg/mL) delivered as two injections of 2 mL each and administered subcutaneously on opposite sides of the abdomen. Each vial of the Leronlimab (PRO 140) product may contain ˜1.4 mL antibody at a concentration of 175 mg/mL.

In embodiment, described herein, a therapeutic agent that is not a CCR5 binding agent may be administered in conventional doses using conventional methods. In another embodiment, a therapeutic agent that is not a CCR5 binding agent may be administered in lower doses due to synergistic effects achieved by administration of the CCR5 binding agent.

A CCR5 binding agent, such as leronlimab, may be administered together with a non-CCR5 binding agent at the same time or in serial order. Administration together may be effectively achieved wherein a subject experiences therapeutic effect for each of the CCR5 binding agent and the non-CCR5 binding agent regardless of the particular dosing regimen or order of introduction of the therapeutically effective agents.

In any of the aforementioned embodiments, the cancer may be, for example, breast cancer, prostate cancer, colon cancer, melanoma, gastric cancer, ovarian cancer, lung (non-small cell) cancer, pancreatic cancer, sarcoma, or blood cell cancer. In a particular embodiment, the cancer is breast cancer. In a particular embodiment, the cancer is metastatic breast cancer.

### Methods of Treating Metastatic Breast Cancer

In one aspect, the present disclosure provides methods of treating or preventing metastatic breast cancer comprising administering to a subject in need thereof a CCR5 binding agent in combination with another therapeutic agent.

In particular embodiments, the methods disclosed herein comprise administering leronlimab in combination with a DNA damaging agent, such as, for example, doxorubicin or carboplatin.

In one embodiment, the competitive inhibitor to a CCR5 cell receptor, such as PRO 140, is administered in combination with one or more DNA damaging agents, such as chemotherapeutics which may include but are not limited to: alkylating agents such as thiotepa and cyclosphosphamide; alkyl sulfonates such as busulfan, improsulfan, and piposulfan; aziridines such as benzodopa, carboquone, meturedopa, and uredopa; ethylenimines and methylamelamines including altretamine, triethylenemelamine, trietylenephosphoramide, triethylenethiophosphaoramide, and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas such as carmustine, chlorozotocin, fotemustine, lomustine, nimustine, and ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin; anti-metabolites such as methotrexate and 5-fluorouracil (5-FU); folic acid analogues such as denopterin, methotrexate, pteropterin, and trimetrexate; purine analogs such as fludarabine, 6-mercaptopurine, thiamiprine, and thioguanine; pyrimidine analogs such as ancitabine, azacitidine, 6-azauridine, carmofur, cytarabine, dideoxyuridine, doxifluridine, enocitabine, floxuridine, and 5-FU; androgens such as calusterone, dromostanolone propionate, epitiostanol, mepitiostane, and testolactone; anti-adrenals such as aminoglutethimide, mitotane, and trilostane; folic acid replenishers such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK™; razoxane; sizofiran; spirogermanium; tenuazonic acid; triaziquone; 2, 2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g., paclitaxel (Taxol™, Bristol-Myers Squibb Oncology, Princeton, N.J.) and docetaxel (Taxotere™, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; and capecitabine; and pharmaceutically acceptable salts, acids, or derivatives of any of the above.

In particular embodiments, the metastatic breast cancer comprises metastatic triple negative breast cancer and the method comprises administering leronlimab in combination with doxorubicin, or leronlimab in combination with carboplatin.

In one embodiment, the present disclosure provides a method of treating or preventing CCR5 positive metastatic breast cancer comprising administering to a subject in need thereof an effective amount of a CCR5 binding agent.

In a further embodiment, the CCR5 binding agent competes with CCL5 for binding to the CCR5 cell receptor. In a further embodiment, the CCR5 binding agent comprises the monoclonal antibody PA14, leronlimab, or CCR5mAb004, or a binding fragment thereof. In a further embodiment, the competitive inhibitor competes for binding with the monoclonal antibody PA14, leronlimab, or CCR5mAb004, or a binding fragment thereof.

In one embodiment, the present disclosure provides a method of treating or preventing CCR5 positive metastatic breast cancer comprising administering to a subject in need thereof leronlimab, or binding fragment thereof.

In any of the aforementioned embodiments, preventing the metastatic breast cancer may comprise slowing the growth or spread of the cancer metastasis or the primary tumor, preventing the formation of a metastatic tumor, or limiting or reducing the growth or size of a metastatic tumor or primary tumor.

In one embodiment, CCR5 binding agent, such as leronlimab, is administered with a pharmaceutically acceptable carrier. Pharmaceutically acceptable carriers are well known to those skilled in the art. Such pharmaceutically acceptable carriers may include but are not limited to aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, saline, and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, chelating agents, inert gases, and the like. In one embodiment, the CCR5 binding agent is provided in a formulation as disclosed in U.S. Pat. No. 9,956,165, the contents of which are incorporated here by this reference.

The dose of the composition of the disclosure will vary depending on the subject and upon the particular route of administration used. Dosages can range from 0.1 to 100,000 μg/kg. Based upon the composition, the dose can be delivered continuously, such as by continuous pump, or at periodic intervals, e.g., on one or more separate occasions. Desired time intervals of multiple doses of a particular composition can be determined without undue experimentation by one skilled in the art.

In one embodiment of the instant methods, the antibody or binding fragment thereof is administered to the subject a plurality of times and each administration delivers from 0.01 mg per kg body weight to 50 mg per kg body weight of the antibody or binding fragment thereof to the subject. In another embodiment, each administration delivers from 0.05 mg per kg body weight to 25 mg per kg body weight of the antibody or binding fragment thereof to the subject. In a further embodiment, each administration delivers from 0.1 mg per kg body weight to 10 mg per kg body weight of the antibody or binding fragment thereof to the subject. In a still further embodiment, each administration delivers from 0.5 mg per kg body weight to 5 mg per kg body weight of the antibody or binding fragment thereof to the subject. In another embodiment, each administration delivers from 1 mg per kg body weight to 3 mg per kg body weight of the antibody or binding fragment thereof to the subject. In another embodiment, each administration delivers about 2 mg per kg body weight of the antibody or binding fragment thereof to the subject. Embodiments include dosages in amounts ranging from about 175 mg to about 1,400 mg, including dosage forms delivering certain amounts of the CCR5 binding agent such as 175 mg, 350 mg, 525 mg, 700 mg, 875 mg, 1050 mg, 1,225 mg, and 1,400 mg.

In one embodiment, the antibody or binding fragment thereof is administered a plurality of times, and a first administration is separated from the subsequent administration by an interval of less than one week. In another embodiment, the first administration is separated from the subsequent administration by an interval of at least one week. In a further embodiment, the first administration is separated from the subsequent administration by an interval of one week. In another embodiment, the first administration is separated from the subsequent administration by an interval of two to four weeks. In another embodiment, the first administration is separated from the subsequent administration by an interval of two weeks. In a further embodiment, the first administration is separated from the subsequent administration by an interval of four weeks. In yet another embodiment, the antibody or binding fragment thereof is administered a plurality of times, and a first administration is separated from the subsequent administration by an interval of at least one month.

In a further embodiment, the antibody or binding fragment thereof is administered to the subject via intravenous infusion. In another embodiment, the antibody or binding fragment thereof is administered to the subject via subcutaneous injection. In another embodiment, the antibody or binding fragment thereof is administered to the subject via intramuscular injection.

In one embodiment, the aforementioned methods may further comprise administering to the subject a cellular therapy, e.g., an autologous or allogeneic immunotherapy; a small molecule; a chemotherapeutic agent; or an inhibitor of CCR5/CCL5 signaling. In one embodiment, an inhibitor of CCR5/CCL5 signaling is administered, and comprises maraviroc, vicriviroc, aplaviroc, SCH-C, TAK-779, PA14 antibody, 2D7 antibody, RoAb13 antibody, RoAb14 antibody, or 45523 antibody.

In one embodiment, the competitive inhibitor to a CCR5 cell receptor, such as PRO 140, is administered in combination with one or more other therapeutic molecules or treatment, such a cellular therapy, e.g., an autologous or allogeneic immunotherapy; a small molecule; a chemotherapeutic; or an inhibitor of CCR5/CCL5 signaling, such as maraviroc, vicriviroc, aplaviroc, SCH-C, TAK-779, PA14 antibody, 2D7 antibody, RoAb13 antibody, RoAb14 antibody, or 45523 antibody. In one embodiment, the methods disclosed herein comprise administering PRO 140 in combination with maraviroc, vicriviroc, aplaviroc, SCH-C, TAK-779, PA14 antibody, 2D7 antibody, RoAb13 antibody, RoAb14 antibody, or 45523 antibody.

In one embodiment, the CCR5 binding agent, such as PRO 140, is administered alongside one or more chemotherapeutics such as, for example: alkylating agents such as thiotepa and cyclosphosphamide; alkyl sulfonates such as busulfan, improsulfan, and piposulfan; aziridines such as benzodopa, carboquone, meturedopa, and uredopa; ethylenimines and methylamelamines including altretamine, triethylenemelamine, trietylenephosphoramide, triethylenethiophosphaoramide, and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas such as carmustine, chlorozotocin, fotemustine, lomustine, nimustine, and ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin; anti-metabolites such as methotrexate and 5-fluorouracil (5-FU); folic acid analogues such as denopterin, methotrexate, pteropterin, and trimetrexate; purine analogs such as fludarabine, 6-mercaptopurine, thiamiprine, and thioguanine; pyrimidine analogs such as ancitabine, azacitidine, 6-azauridine, carmofur, cytarabine, dideoxyuridine, doxifluridine, enocitabine, floxuridine, and 5-FU; androgens such as calusterone, dromostanolone propionate, epitiostanol, mepitiostane, and testolactone; anti-adrenals such as aminoglutethimide, mitotane, and trilostane; folic acid replenishers such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK™; razoxane; sizofiran; spirogermanium; tenuazonic acid; triaziquone; 2, 2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g., paclitaxel (Taxol™, Bristol-Myers Squibb Oncology, Princeton, N.J.) and docetaxel (Taxotere™, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; and capecitabine; and pharmaceutically acceptable salts, acids, or derivatives of any of the above.

As used herein, a “small-molecule” CCR5 receptor antagonist includes, for example, a small organic molecule which binds to a CCR5 receptor and inhibits the activity of the receptor. In one embodiment, the small molecule has a molecular weight less than 1,500 daltons. In another embodiment, the small molecule has a molecular weight less than 600 daltons.

In one embodiment, the CCR5 binding agent, such as PRO 140, is administered in combination with one or more small molecules, such as SCH-C(Strizki et al., PNAS, 98: 12718-12723 (2001)); SCH-D (SCH 417670; vicriviroc); UK-427,857 (maraviroc; 1-[(4,6-dimethyl-5-pyrimidinyl) carbonyl]-4-[4-[2-methoxy-1(R)-4-(trifluoromethyl)phenyl]ethyl-3(S)-methyl-1-piperazinyli-4-methylpiperidine); GW873140; TAK-652; TAK-779; AMD070; AD101; 1,3,4-trisubstituted pyrrolidines (Kim et al., Bioorg. Med. Chem. Lett., 15: 2129-2134 (2005)); modified 4-piperidinyl-2-phenyl-1-(phenylsulfonylamino)-butanes (Shah et al., Bioorg. Med. Chem. Lett., 15: 977-982 (2005)); Anibamine TFA, Ophiobolin C, or 19,20-epoxycytochalasin Q (Jayasuriya et al., J. Nat. Prod., 67: 1036-1038 (2004)); 5-(piperidin-1-yl)-3-phenyl-pentylsulfones (Shankaran et al., Bioorg. Med. Chem. Lett., 14: 3589-3593 (2004)); 4-(heteroarylpiperdin-1-yl-methyl)-pyrrolidin-1-yl-acetic acid antagonists (Shankaran et al., Bioorg. Med. Chem. Lett., 14: 3419-3424 (2004)); agents containing 4-(pyrazolyl)piperidine side chains (Shu et al., Bioorg. Med. Chem. Lett., 14: 947-52 (2004); Shen et al., Bioorg. Med. Chem. Lett., 14: 935-939 (2004); Shen et al., Bioorg. Med. Chem. Lett., 14: 941-945 (2004)); 3-(pyrrolidin-1-yl)propionic acid analogues (Lynch et al., Org. Lett., 5: 2473-2475 (2003)); [2-(R)-[N-methyl-N-(1-(R)-3-(S)-((4-(3-benzyl-1-ethyl-(1H)-pyrazol-5-yl)piperidin-1-yl)methyl)-4-(S)-(3-fluorophenyl)cyclopent-1-yl)amino]-3-methylbutanoic acid (MRK-1)] (Kumar et al., J. Pharmacol. Exp. Ther., 304: 1161-1171 (2003)); 1,3,4 trisubstituted pyrrolidines bearing 4-aminoheterocycle substituted piperidine side chains (Willoughby et al., Bioorg. Med. Chem. Lett., 13: 427-431 (2003); Lynch et al., Bioorg. Med. Chem. Lett., 12: 3001-3004 (2003); Lynch et al., Bioorg. Med. Chem. Lett., 13: 119-123 (2003); Hale et al., Bioorg. Med. Chem. Lett., 12: 2997-3000 (2002)); bicyclic isoxazolidines (Lynch et al., Bioorg. Med. Chem. Lett., 12: 677-679 (2002)); combinatorial synthesis of CCR5 antagonists (Willoughby et al., Bioorg. Med. Chem. Lett., 11: 3137-41 (2001)); heterocycle-containing compounds (Kim et al., Bioorg. Med. Chem. Lett., 11: 3103-3106 (2001)); antagonists containing hydantoins (Kim et al., Bioorg. Med. Chem. Lett., 11: 3099-3102 (2001)); 1,3,4 trisubstituted pyrrolidines (Hale et al., Bioorg. Med. Chem. Lett., 11: 2741-2745 (2001)); 14N-(methyl)-N-(phenylsulfonyl)amino]-2-(phenyl)-4-(4-(N-(alkyl)-N-(benzyloxycarbonyl)amino)piperidin-1-yl)butanes (Finke et al., Bioorg. Med. Chem. Lett., 11: 2475-2479 (2001)); compounds from the plant Lippia alva (Hedge et al., Bioorg. Med. Chem. Lett., 12: 5339-5342 (2004)); piperazine-based CCR5 antagonists (Tagat et al., J. Med. Chem., 47: 2405-2408 (2004)); oximino-piperidino-piperidine-based CCR5 antagonists (Palani et al., Bioorg. Med. Chem. Lett., 13: 709-712 (2003)); rotamers of SCH 351125 (Palani et al., Bioorg. Med. Chem. Lett., 13: 705-708 (2003)); piperazine-based symmetrical heteroaryl carboxamides (McCombie et al., Bioorg. Med. Chem. Lett., 13: 567-571 (2003)); oximino-piperidino-piperidine amides (Palani et al., J. Med. Chem., 45: 3143-3160 (2002)); Sch-351125 and Sch-350634 (Este, Curr. Opin. Investig. Drugs., 3: 379-383 (2002)); 1-[(2,4-dimethyl-3-pyridinyl)carbonyl]-4-methyl-4-[3(S)-methyl-4-[1(S)-[4-(trifluoromethyl)phenyl]ethyl]-1-piperazinyl]-piperidine N1-oxide (Sch-350634) (Tagat et al., J. Med. Chem., 44: 3343-3346 (2001)); 4-[(Z)-(4-bromophenyl)-(ethoxyimino)methyl]-1′-[(2,4-dimethyl-3-pyridinyl)carbonyl]-4′-methyl-1,4′-bipiperidine N-oxide (SCH 351125) (Palani et al., J. Med. Chem., 44: 3339-3342 (2001)); 2(S)-methyl piperazines (Tagat et al., Bioorg. Med. Chem. Lett., 11: 2143-2146 (2001)); piperidine-4-carboxamide derivatives (Imamura et al., Bioorg. Med. Chem., 13: 397-416, 2005); 1-benzazepine derivatives containing a sulfoxide moiety (Seto et al., Bioorg. Med. Chem. Lett., 13: 363-386 (2005)); anilide derivatives containing a pyridine N-oxide moiety (Seto et al., Chem. Pharm. Bull. (Tokyo), 52: 818-829 (2004)); 1-benzothiepine 1,1-dioxide and 1-benzazepine derivatives containing a tertiary amine moiety (Seto et al., Chem. Pharm. Bull. (Tokyo), 52: 577-590 (2004)); N-[3-(4-benzylpiperidin-1-yl)propyl]-N,N′-diphenylureas (Imamura et al., Bioorg. Med. Chem., 12: 2295-2306 (2004)); 5-oxopyrrolidine-3-carboxamide derivatives (Imamura et al., Chem. Pharm. Bull. (Tokyo), 52: 63-73 (2004); anilide derivatives with a quaternary ammonium moiety (Shiraishi et al., J. Med. Chem., 43: 2049-2063 (2000)); AK602/0N04128/GW873140 (Nakata et al., J. Virol., 79: 2087-2096 (2005)); spirodiketopiperazine derivatives (Maeda et al., J. Biol. Chem., 276: 35194-35200 (2001); Maeda et al., J. Virol., 78: 8654-8662 (2004)); and selective CCR5 antagonists (Thoma et al., J. Med. Chem., 47: 1939-1955 (2004)).

In one embodiment, the CCR5 binding agent, such as PRO 140, is administered in combination with one or more of SCH-C, SCH-D (SCH 417670, or vicriviroc), UK-427,857 (maraviroc), GW873140, TAK-652, TAK-779 AMD070, or AD101. See U.S. Pat. No. 8,821,877.

In one embodiment, the competitive binding agent to a CCR5 cell receptor, such as PRO 140, exhibits synergistic effects when administered in combination with a DNA damaging agent. In further embodiments, the competitive binding agent to a CCR5 cell receptor, such as PRO 140, exhibits synergistic effects when administered in combination with a DNA damaging agent and along side one or more other therapeutic molecules or treatment, such as a cellular therapy, a small molecule, a chemotherapeutic, or an inhibitor of CCR5/CCL5 signaling.

“Synergy” between two or more agents refers to the combined effect of the agents which is greater than their additive effects. Synergistic, additive, or antagonistic effects between agents may be quantified by analysis of the dose-response curves using the Combination Index (CI) method. A CI value greater than 1 indicates antagonism; a CI value equal to 1 indicates an additive effect; and a CI value less than 1 indicates a synergistic effect. In one embodiment, the CI value of a synergistic interaction is less than 0.9. In another embodiment, the CI value is less than 0.8. In another embodiment, the CI value is less than 0.7.

In any of the aforementioned embodiments, preventing the cancer may comprise slowing the growth of the cancer, preventing the formation of a tumor, or limiting or reducing the growth or size of a tumor.

In one embodiment, the competitive inhibitor to a CCR5 cell receptor, such as PRO 140, is administered with a pharmaceutically acceptable carrier. Pharmaceutically acceptable carriers are well known to those skilled in the art. Such pharmaceutically acceptable carriers may include but are not limited to aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, saline, and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, chelating agents, inert gases, and the like.

The dose of the composition of the disclosure will vary depending on the subject and upon the particular route of administration used. Dosages can range from 0.1 to 100,000 μg/kg. Based upon the composition, the dose can be delivered continuously, such as by continuous pump, or at periodic intervals, e.g., on one or more separate occasions. Desired time intervals of multiple doses of a particular composition can be determined without undue experimentation by one skilled in the art.

In one embodiment of the instant methods, the antibody or binding fragment thereof is administered to the subject a plurality of times and each administration delivers from 0.01 mg per kg body weight to 50 mg per kg body weight of the antibody or binding fragment thereof to the subject. In another embodiment, each administration delivers from 0.05 mg per kg body weight to 25 mg per kg body weight of the antibody or binding fragment thereof to the subject. In a further embodiment, each administration delivers from 0.1 mg per kg body weight to 10 mg per kg body weight of the antibody or binding fragment thereof to the subject. In a still further embodiment, each administration delivers from 0.5 mg per kg body weight to 5 mg per kg body weight of the antibody or binding fragment thereof to the subject. In another embodiment, each administration delivers from 1 mg per kg body weight to 3 mg per kg body weight of the antibody or binding fragment thereof to the subject. In another embodiment, each administration delivers about 2 mg per kg body weight of the antibody or binding fragment thereof to the subject.

In one embodiment, the antibody or binding fragment thereof is administered a plurality of times, and a first administration is separated from the subsequent administration by an interval of less than one week. In another embodiment, the first administration is separated from the subsequent administration by an interval of at least one week. In a further embodiment, the first administration is separated from the subsequent administration by an interval of one week. In another embodiment, the first administration is separated from the subsequent administration by an interval of two to four weeks. In another embodiment, the first administration is separated from the subsequent administration by an interval of two weeks. In a further embodiment, the first administration is separated from the subsequent administration by an interval of four weeks. In yet another embodiment, the antibody or binding fragment thereof is administered a plurality of times, and a first administration is separated from the subsequent administration by an interval of at least one month.

In a further embodiment, the antibody or binding fragment thereof is administered to the subject via intravenous infusion. In another embodiment, the antibody or binding fragment thereof is administered to the subject via subcutaneous injection. In another embodiment, the antibody or binding fragment thereof is administered to the subject via intramuscular injection.

In one embodiment, the aforementioned methods may further comprise administering to the subject a cellular therapy, e.g., an autologous or allogeneic immunotherapy; a small molecule; a chemotherapeutic agent; or an inhibitor of CCR5/CCL5 signaling. In one embodiment, an inhibitor of CCR5/CCL5 signaling is administered, and comprises maraviroc, vicriviroc, aplaviroc, SCH-C, TAK-779, PA14 antibody, 2D7 antibody, RoAb13 antibody, RoAb14 antibody, or 45523 antibody.

In one embodiment, the competitive inhibitor to a CCR5 cell receptor, such as PRO 140, is administered in combination with one or more other therapeutic molecules or treatment, such a cellular therapy, e.g., an autologous or allogeneic immunotherapy; a small molecule; a chemotherapeutic; or an inhibitor of CCR5/CCL5 signaling, such as maraviroc, vicriviroc, aplaviroc, SCH-C, TAK-779, PA14 antibody, 2D7 antibody, RoAb13 antibody, RoAb14 antibody, or 45523 antibody. In one embodiment, the methods disclosed herein comprise administering PRO 140 in combination with maraviroc, vicriviroc, aplaviroc, SCH-C, TAK-779, PA14 antibody, 2D7 antibody, RoAb13 antibody, RoAb14 antibody, or 45523 antibody.

While the studies described herein relate to the study of breast cancer cell lines, it is contemplated that other cancers expressing CCR5 may also benefit for the use of leronlimab or other anti-CCR5 agents to block metastasis and/or enhance cell death induced by DNA damaging chemotherapy. Such other cancers may include, but are not limited to, one of leukemia cancer, lymphoma cancer, bone and connective tissue sarcoma, brain tumor cancer, breast cancer, adrenal cancer, pancreatic cancer, stomach cancer, colon cancer, prostate cancer, rectal cancer, gallbladder cancer, lung cancer, oral cancer, skin cancer, kidney cancer, and osteogenic sarcoma cancer, and others.

Anti-CCR5 agents may include, but are not limited to, antibodies, other proteins, and small molecule agents such as, for example, maraviroc and vicriviroc.

The DNA damaging chemotherapy agent may include, but is not limited to, anthracyclines doxorubicin, daunorubicin, epirubicin, idarubicin, mitoxantrone, and ametantrone, and any derivatives thereof. Other chemotherapy agents may be used with the present disclosure, and may benefit from the present disclosure such as, for example, carboplatin, cisplatin, cyclophosphamide, docetaxel, erlotinib, etoposide, fluorouracil, gemcitabine, imatinib mesylate, irinotecan, methotrexate, paclitaxel, sorafinib, sunitinib, topotecan, vincristine or vinblastine, and others. It is contemplated that any conventional therapeutic agents may be used together with the present disclosure, and may benefit from the present disclosure for use in treating cancer, or cancer metastasis.

### DNA Damaging Agents

Without being bound by theory, it is believed that exposure of cancer cells to certain DNA-damaging agent results in sufficient DNA damage to trigger the DNA damage response and temporary S phase arrest to allow for DNA repair. The DNA damage response is believed to be regulated by two homologous protein kinases, ataxia telangiectasia (ATM) and ataxia telangiectasia Rad3-related (ATR). ATR signals to regulate DNA replication, cell cycle transitions, and DNA repair through the phosphorylation of hundreds of substrates, including checkpoint kinase 1 (Chkl). DNA-damaging agents have a long history of use in cancer chemotherapy. DNA damage induces apoptosis of cells and is widely believed to be the major antiproliferative mechanism of DNA damaging anticancer drugs.

### EXAMPLES

### Example 1. The Binding of Leronlimab With CCR5 Expressed in Breast Cancer Cells

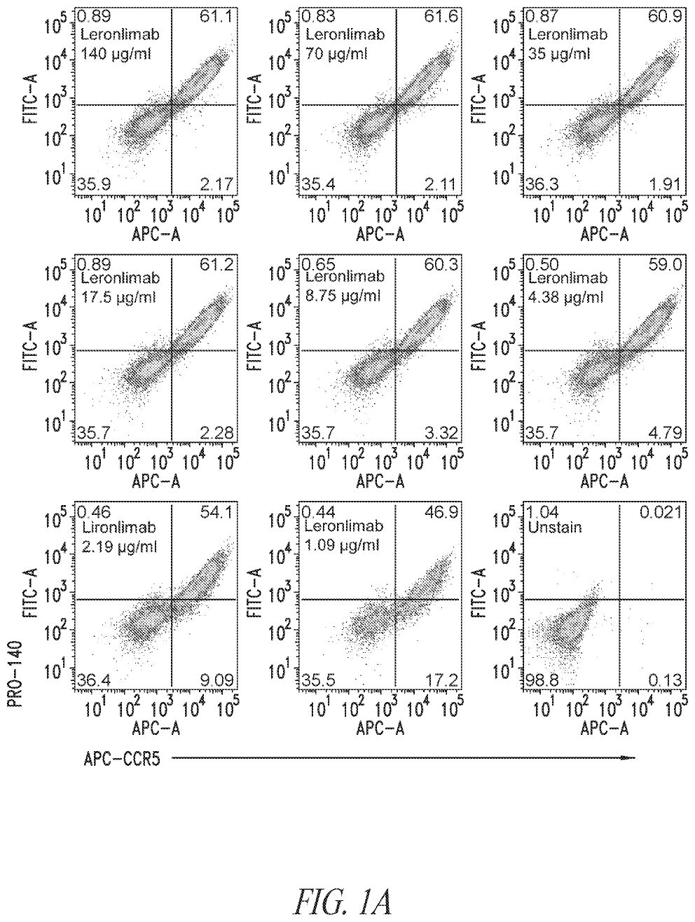

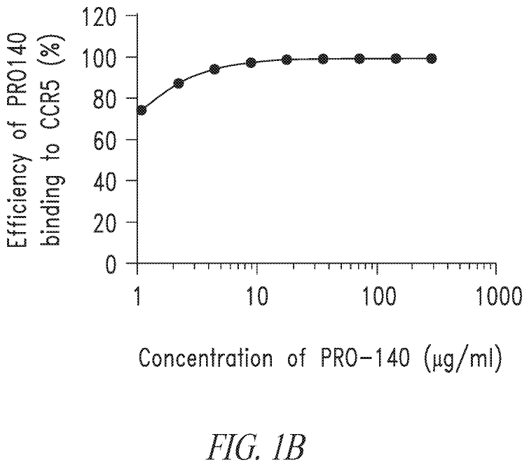

FIGS. 1A and 1B. Leronlimab binds CCR5 in human breast cancer cells.

As shown in FIG. 1A, in order to determine the binding of Leronlimab to human CCR5 in breast cancer cells, a MDA-MB-231 human breast cancer cell line was transfected with a human CCR5 expression vector as a model system. A previously tested commercial APC conjugated mouse anti-human/mouse/rat CCR5 antibody from R&D (FAB1802A) (APC-αCCR5) was used as a positive control to assess CCR5 positive cells. MDA-MB-231-CCR5 cells were stained with both APC-αCCR5 and leronlimab using the concentration from 1-140 □g/ml. Alexa Fluor 488 conjugated mouse anti-human IgG was used as secondary antibody to measure leronlimab binding cells. Analysis of leronlimab binding with CCR5 by FACS is shown in FIG. 1A. Leronlimab binding with human CCR5 was validated.

As shown in FIG. 1B, the efficiency of PRO140 binding to CCR5 positive cells was up to 98%.

### Example 2. The Effects of PRO140 on CCL5 Induced Ca²⁺ Responses in MDA-MB-231-CCR5 Cells

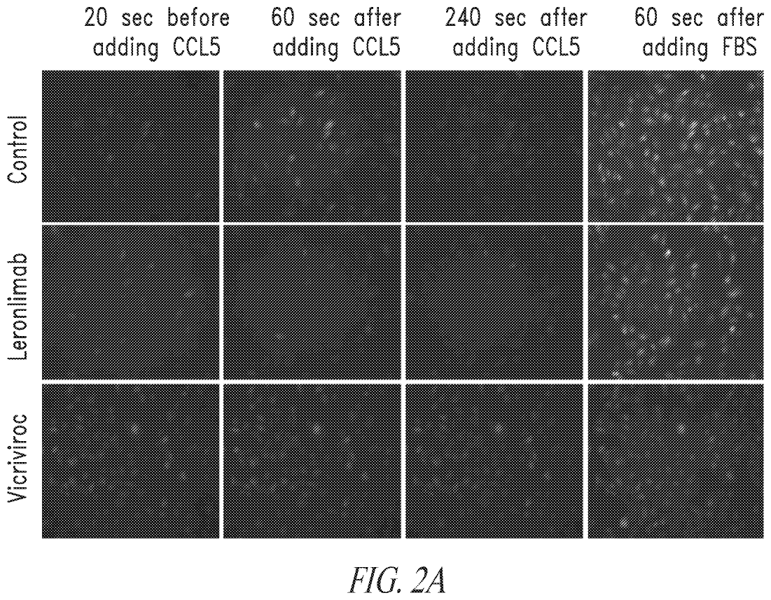

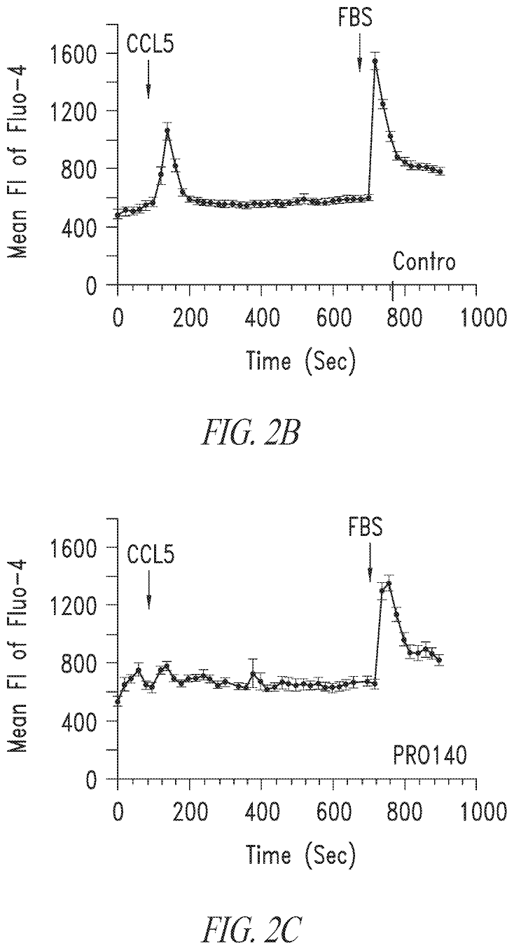

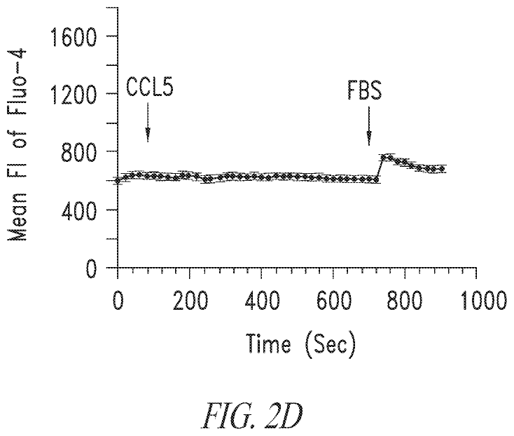

FIG. 2A, FIG. 2B, FIG. 2C, and FIG. 2D. PRO140 (leronlimab) blocks human CCR5-mediated signaling in human breast cancer cells. CCR5 activation induces calcium flux (Mueller et al., 2002; Petkovic et al., 2004). To assess the effects of leronlimab on CCR5 function, we measured the calcium responses induced by CCL5 in MDA-MB-231-CCR5 cells with or without leronlimab by living cell image (FIG. 2A, FIG. 2B, and FIG. 2C). Fluo-4 was used as calcium concentration indicator. The CCR5 antagonist, vicriviroc, was used as positive control (FIGS. 2A and 2D). The results showed that leronlimab can block CCL5 induced calcium responses in MDA-MB-231-CCR5 cells (1.23±0.10, N=10 for control cells and 0.54±0.13 N=12 for PRO140 treated cells. P<0.001 at calcium peak induce by CCL5).

### Example 3. Leronlimab Blocks Breast Cancer Cell 3D-Matrigel Invasion

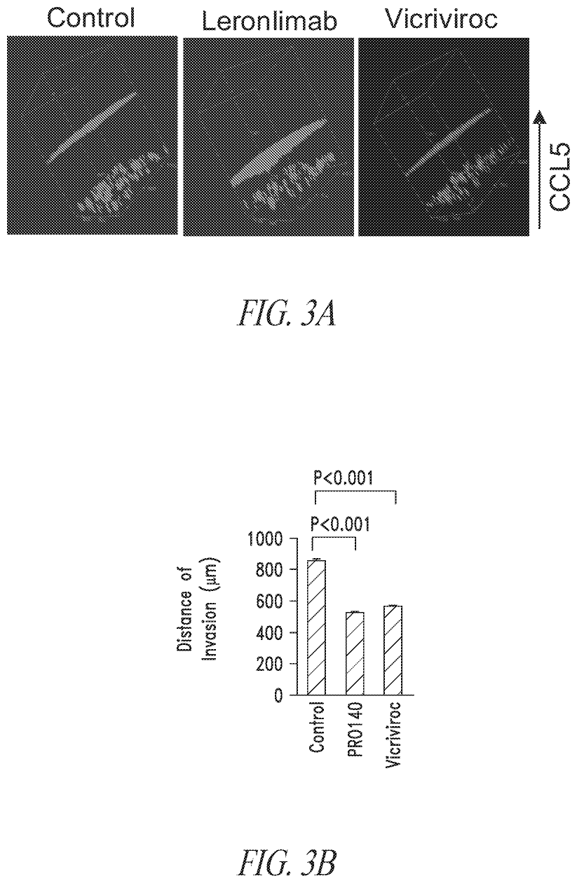

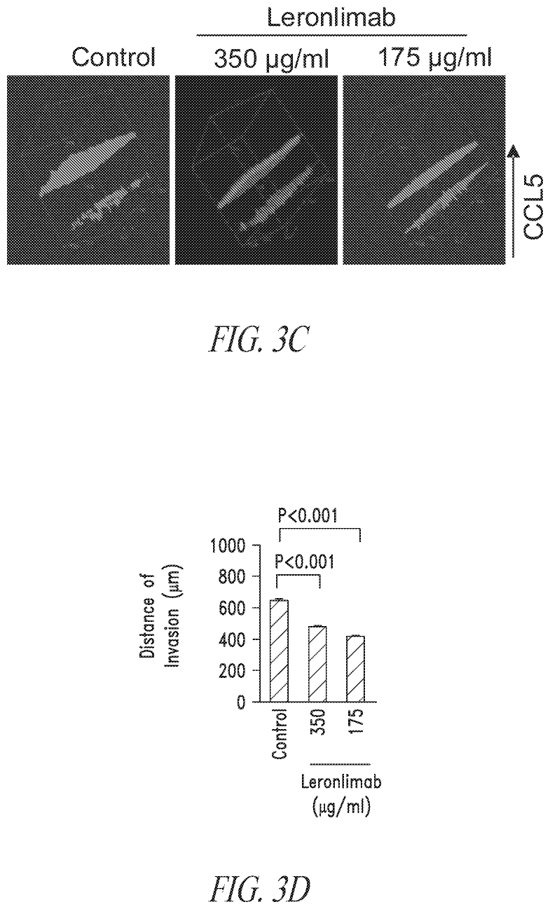

FIG. 3A, FIG. 3B, FIG. 3C, and FIG. 3D. Leronlimab blocks CCR5 mediated invasion of human breast cancer cells into extracellular matrix. The ability of breast cancer cells to invade extra-cellular matrix is distinguishable from but an important step in tumor metastasis (Zetter, 1990). To test the ability of PRO140 to block 3D-matrigel invasion assay, MDA-MB-231 cells were used. CCL5 was used as chemoattractant to induce invasion. The small molecule inhibitor of CCR5, vicriviroc, was used as a form of positive control. Leronlimab reduced CCL5-induced MDA-MB-231 breast cancer cell invasion with similar efficacy as vicriviroc (FIG. 3A, FIG. 3B) (855±9, N=8 for control vs 855±9, N=9 for leronlimab, P<0.001). We also tested the effects of different dose of leronlimab on breast cancer cell invasion and the results showed that both 175 and 350 □g/ml of leronlimab can effectively block MDA-MB-231 cell invasion (FIG. 3C, FIG. 3D).

### Example 4. Leronlimab Blocks Breast Cancer Cell Metastasis in a Mouse Lung Metastasis Model

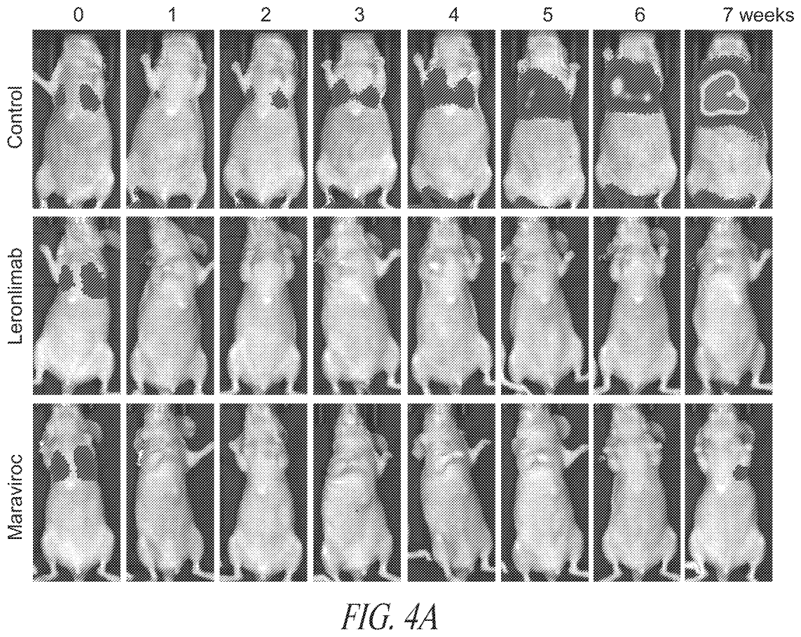

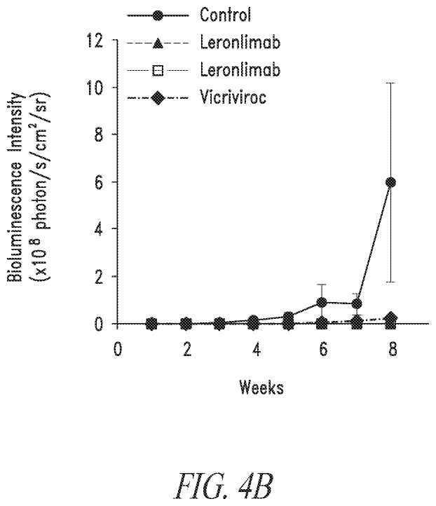

FIG. 4A and FIG. 4B. Leronlimab block breast cancer metastasis in mice. The mice were divided into 4 groups (control, leronlimab, maraviroc and vicriviroc) randomly. MDA-MB-231 cells stable transfected with Luc2-GFP was injected into the mice through tail-vein. The mice in each group were treated one day before injection. The metastasis tumor formed in the lung was determined by bioluminescence imaging. The bioluminescence images of the representative mice from control, Leronlimab and Maraviroc group were showed in (FIG. 4A). The quantitative analysis of tumor size in each group was shown in (FIG. 4B). The size of tumors defined by photon flux (x10⁸ p/sec/cm²/sr). The data was showed as Mean±SE. Leronlimab dramatically decreased breast cancer tumor metastasis to the lung.

### Example 5. Leronlimab Enhances Cell Death Induced by Doxorubicin

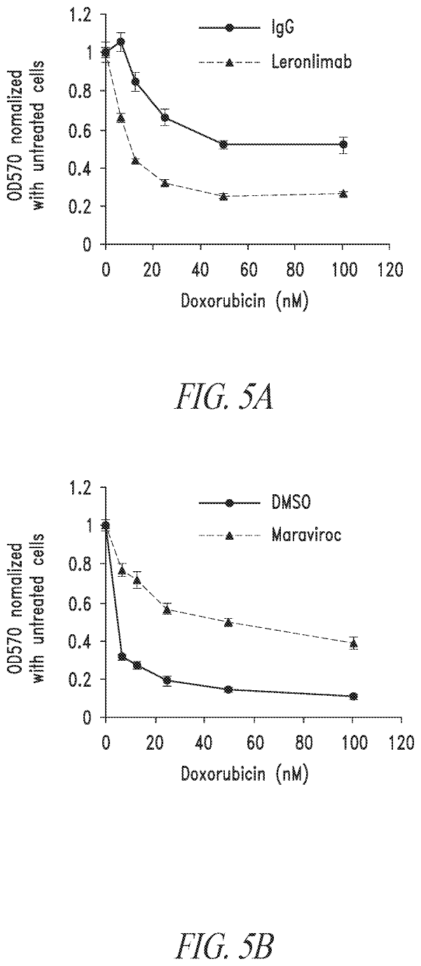

FIG. 5A and FIG. 5B. Leronlimab enhances the cell death induced by doxorubicin, a DNA damage inducing chemotherapy agent. MDA-MB-231 cells were treated with 10 mg/ml of leronlimab combining with different dose of doxorubicin for 3 days. The MTT assay was used to determine the relative cell number (FIG. 5A). The cells treated with maraviroc (100 mM) combining with different dose of doxorubicin were used as positive control (FIG. 5B). Data are shown as mean±SEM for N=8.

### Example 6. Leronlimab and Carboplatin Treatment of CCR5+ Metastatic TNBC

Described here are interim results from a phase Ib/II study of leronlimab (PRO 140) combined with carboplatin in patients with CCR5+ metastatic Triple Negative Breast Cancer (mTNBC). The primary objective of Phase 1b is to determine the safety, tolerability and maximum tolerate dose (MTD) of PRO 140 in patients with TNBC, when combined with carboplatin to define a recommended Phase II dose of the combination. The primary objective of phase 2b is to evaluate the impact on progression-free survival (PFS) of the combination PRO 140 and carboplatin in patients with CCR5+ TNBC previously treated with anthracyclines and taxanes in neoadjuvant and adjuvant setting.

A first subject enrolled in the study, subject 706-001, is a 42 year old female with Stage IV metastatic triple negative breast cancer. Subject has a history of left breast cancer with a right lung metastasis.

The subject was diagnosed with Stage IIA Grade 3 Invasive Ductal Carcinoma (ER neg/PR neg/HER-2-NEU neg. and previously received dose-dense Adriamycin (Doxorubicin) and Cyclophosphamide [ddAC] and Paclitaxel. The subject underwent a left lumpectomy of the breast and a sentinel lymph node biopsy three weeks following diagnosis.

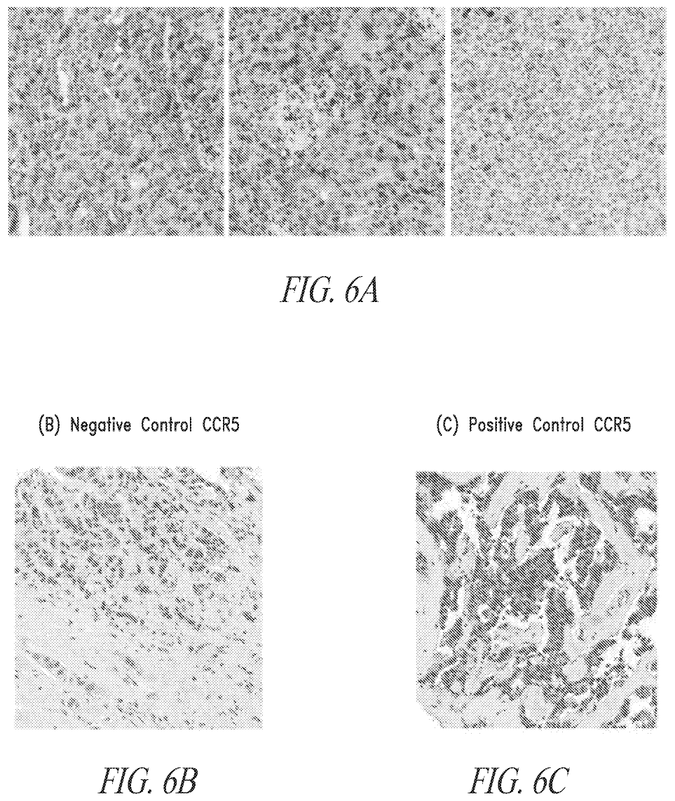

The subject signed the pre-screening informed consent for the Protocol CD07_TNBC ten weeks following diagnosis. The immunohistochemistry analysis on archival tissue showed high predominance of CCR5+ tumor infiltrating leukocytes (FIGS. 6A-6C).

The baseline target lesion was identified in the right upper lung at the size of 25 mm. The lesion was described as pleural based, major fissure, soft tissue density nodule in the right hilum.

Approximately six weeks following the identification and measurement of the baseline lesion, the subject received the first treatment of 350 mg leronlimab (PRO 140) (1). Each treatment cycle consisted of 21 days. Leronlimab (PRO 140) was administered subcutaneously weekly on Days 1, 8, and 15 in combination with carboplatin AUC 5 on Day 1 of each cycle (every 21 days). This treatment regimen was used for all subjects enrolled in the mTNBC study, unless otherwise indicated.

TABLE 1
Leronlimab (PRO 140) and
Carboplatin Doses Subject 706-001
Visit Study Treatment Administration

| Pre-Screening | NA | |
|---|---|---|
| Screening | NA | |
| NA | Carboplatin | 500 mg |
| C1D1 | Leronlimab (PRO 140) | 350 mg |
| C1D8 | Leronlimab (PRO 140) | 350 mg |
| C1D15 | Leronlimab (PRO 140) | 350 mg |
| C2D1 | Leronlimab (PRO 140) | 350 mg |
| | Carboplatin | 500 mg |
| C2D8 | Leronlimab (PRO 140) | 350 mg |
| C2D15 | Leronlimab (PRO 140) | 350 mg |
| C3D1 | Leronlimab (PRO 140) | 350 mg |
| | Carboplatin | 500 mg |
| C3D8 | Leronlimab (PRO 140) | 350 mg |
| C3D15 | Leronlimab (PRO 140) | 350 mg |
| C4D1 | Leronlimab (PRO 140) | 350 mg |
| | Carboplatin | 250 mg |
| C4D8 | Leronlimab (PRO 140) | 350 mg |
| C4D15 | Leronlimab (PRO 140) | 350 mg |
| C5D1 | Leronlimab (PRO 140) | 350 mg |
| | Carboplatin | 600 mg |
| C5D8 | Leronlimab (PRO 140) | 350 mg |
| C5D15 | Leronlimab (PRO 140) | 350 mg |
| C6D1 | Leronlimab (PRO 140) | 350 mg |
| | Carboplatin* | |

*Pending dose information

The blood sample for circulating tumor cells (CTC) and cancer associated macrophage-like cells (CAMLs) assessment was collected at baseline and subsequently at Day 1 of each treatment cycle to assess changes in CTCs and CAMLs after treatment and to perform correlative analysis between CCR5 expression and PD-L1 expression.

Creatv Microtech has developed a size based technology and detection methodology (LifeTrac Assay) that enables the collection and characterization of all cancer associated cells in the blood i.e., CTCs, epithelial mesenchymal transition cells (EMTs) and CAMLs.[Adams Cytometry 2015, Adams RSC 2014]. The CellSieve™ filtration platform is used to capture CAMLs and CTCs.

The summary of results for CCR5 expression and PD-L1 expression is as follows:

TABLE 2
Subject 706-001-CCR5-expressing and PD-L1-
expressing CTCs, EMTs, and CAMLs.

| CCR5 | Baseline | C1D1 | C2D1 | C3D1 | C4D1 | C5D1 |
|---|---|---|---|---|---|---|
| Number of CTCs | 1 | 0 | 0 | 0 | 0 | 0 |
| Number of Apoptotic CTCs | 1 | 0 | 0 | 0 | 0 | 0 |
| Number of EMTs | 1 | 1 | 0 | 0 | 0 | 0 |
| Number of CAMLs | 1 | 0 | 1 | 3 | 0 | 1 |
| Largest CAML | 30 μm | NA | 27 μm | 39 μm | 0 μm | 33 μm |
| PD-L1 | Baseline | C1D1 | C2D1 | C3D1 | C4D1 | C5D1 |
| Number of CTCs | 0 | 0 | 0 | 0 | 0 | 0 |
| Number of Apoptotic CTC | 3 | 0 | 0 | 0 | 0 | 0 |
| Number of EMTs | 1 | 1 | 0 | 0 | 0 | 0 |
| Number of CAMLs | 1 | 1 | 2 | 1 | 1 | 2 |
| Largest CAML | 50 μm | 47 μm | 69 μm | 30 μm | 31 μm | 56 μm |

The summary for results of total CTCs, EMTs, and CAMLs is as follows:

TABLE 3
Subject 706-001-CTCs, EMTs, and CAMLs Results

| | Baseline | C1D1 | C2D1 | C4D1 | C5D1 | C6D1 |
|---|---|---|---|---|---|---|
| CTC-Total | 5 | 0 | 0 | 0 | 0 | 0 |
| EMT-Total | 2 | 2 | 0 | 0 | 0 | 0 |
| CAML-Total | 2 | 1 | 3 | 1 | 3 | 8 |

Scans were taken at the end of every two cycles (every 6 weeks). The subject had Scan 1 after six weeks, a Scan 2 after 12 weeks, and Scan 3 after 18 weeks (Table 4). At scan 3, there were no new lung nodules found. The target lesion found on the right upper lobe of the lung nodule measured 2.1×1.6 cm, which was previously 2.4×1.9, had a 20% decrease in size.

TABLE 4
Subject 706-001-Tumor imaging
Subject 706-001
Target Lesion
(Right Upper Lobe lung nodule)
Baseline Scan 25 mm
Scan 2 2.4 × 1.9 cm
Scan 3 2.1 × 1.6 cm

Right lung metastasis demonstrates maximum standardized uptake values (SUVs) of 6.8 (previously 15.3). Previously identified right hilar lymph node resolved. No new lymphadenopathy or metastatic disease reported on the diagnostic CT chest, abdomen and pelvis.

At the time that the subject had completed the Cycle 6 Day 1 visit, the subject had been receiving weekly injections of leronlimab (PRO 140) and a carboplatin infusion every three weeks per protocol. At the time of the Cycle 6 Day 1 visit, no serious adverse events had been reported.

Following 16 weeks of leronlimab treatment of the first subject enrolled under the mTNBC study showed no detectable circulating tumor cells (CTC) or putative metastatic tumor cells in the peripheral blood. Furthermore, the patient had large reductions in CCR5 expression on cancer-associated cells after approximately 11 weeks of treatment with leronlimab. Additionally, the target lesion found on the right upper lobe of the lung nodule shows a greater than 20% decrease in size (as measured by tumor volume). This result was a remarkable improvement in disease outcome and demonstrates that leronlimab is a promising adjuvant therapy for the treatment of metastatic triple negative breast cancer.

A second subject with mTNBC was enrolled in the mTNBC study. Data collected from the second patient enrolled in the Company's mTNBC Phase 1b/2 trial showed no detectable levels of CTC after two weeks of treatment with the previously described treatment regimen of leronlimab in combination with carboplatin. This patient also showed a 70% reduction in EMT cells after just two weeks of treatment. Initial data from the second patient in the mTNBC trial indicated the CTC dropped to zero after two weeks of treatment with leronlimab. Additionally, the second patient had an initial CAML count of 45, and following at least two weeks of treatment the CAML count decreased to 30.

A third subject was enrolled in the mTNBC study. CTC+EMT counts were measured at initiation of treatment and two weeks following initiation of treatment with the previously described treatment regimen. The results indicate that the third patient's total CTC+EMT counts decreased by 75% during the first two weeks of treatment.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet including U.S. Provisional Patent Application No. 62/827,729 filed on Apr. 1, 2019, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method of treating or preventing cancer metastasis in a subject having CCR5+ cancer comprising administering an anti-CCR5 agent in combination with a DNA damaging agent.

2. The method of claim 1, wherein the cancer is a CCR5+ cancer selected from at least one of the following: leukemia cancer, lymphoma cancer, bone and connective tissue sarcoma, brain tumor cancer, breast cancer, adrenal cancer, pancreatic cancer, stomach cancer, colon cancer, prostate cancer, rectal cancer, gallbladder cancer, lung cancer, oral cancer, skin cancer, kidney cancer, and osteogenic sarcoma cancer.

3. The method of claim 1, wherein the cancer is breast cancer.

4. The method of claim 1, wherein the cancer is triple negative breast cancer (TNBC).

5. The method of claim 1, wherein the anti-CCR5 agent comprises antibodies or fragments thereof, non-antibody proteins or fragments thereof, and small molecule agents.

6. The method of claim 1, wherein the anti-CCR5 agent comprises an antibody, or a fragment thereof.

7. The method of claim 6, wherein the anti-CCR5 agent is leronlimab, or a fragment thereof.

8. The method of claim 1, wherein the DNA damaging agent comprises, wherein the DNA damaging chemotherapy is selected from one of doxorubicin, carboplatin, daunorubicin, epirubicin, idarubicin, mitoxantrone, and ametantrone, and any derivatives thereof.

9. The method of claim 8, wherein the DNA damaging agent comprises doxorubicin or carboplatin.

10. The method of claim 9, wherein the DNA damaging agent consists of doxorubicin.

11. The method of claim 9, wherein the DNA damaging agent consists of carboplatin.

12. The method of any one of claims 1-11, wherein the subject exhibits a reduction in circulating tumor cells (CTC) or putative metastatic tumor cells in the peripheral blood following treatment.

13. The method of any one of claims 1-11, wherein the subject exhibits reduced CCR5 expression on cancer-associated cells after following treatment.

14. The method of any one of claims 1-11, wherein the subject exhibits decreased volume in tumor size following treatment.

15. The method of any one of claims 1-11, wherein the subjects exhibits enhanced killing of cancer cells by the DNA damaging agent.

16. A method of prolonging the effectiveness of DNA damaging chemotherapy for treatment or prevention of a CCR5+ cancer, comprising administering an anti-CCR5 agent.

17. The method of claim 16, wherein the cancer is a CCR5+ cancer selected from at least one of the following: leukemia cancer, lymphoma cancer, bone and connective tissue sarcoma, brain tumor cancer, breast cancer, adrenal cancer, pancreatic cancer, stomach cancer, colon cancer, prostate cancer, rectal cancer, gallbladder cancer, lung cancer, oral cancer, skin cancer, kidney cancer, and osteogenic sarcoma cancer.

18. The method of claim 16, wherein the cancer is breast cancer.

19. The method of claim 18, wherein the cancer is triple negative breast cancer (TNBC).

20. The method of claim 16, wherein the anti-CCR5 agent comprises antibodies or fragments thereof, non-antibody proteins or fragments thereof, and small molecule agents.

21. The method of claim 16, wherein the anti-CCR5 agent comprises an antibody, or a fragment thereof.

22. The method of claim 21, wherein the anti-CCR5 agent is leronlimab, or a fragment thereof.

23. The method of claim 16, wherein the DNA damaging agent comprises, wherein the DNA damaging chemotherapy is selected from one of doxorubicin, carboplatin, daunorubicin, epirubicin, idarubicin, mitoxantrone, and ametantrone, and any derivatives thereof.

24. The method of claim 23, wherein the DNA damaging agent comprises doxorubicin or carboplatin.

25. The method of claim 24, wherein the DNA damaging agent consists of doxorubicin.

26. The method of claim 24, wherein the DNA damaging agent consists of carboplatin.

27. The method of any one of claims 16-26, wherein the subject exhibits a reduction in circulating tumor cells (CTC) or putative metastatic tumor cells in the peripheral blood following treatment.

28. The method of any one of claims 16-26, wherein the subject exhibits reduced CCR5 expression on cancer-associated cells after following treatment.

29. The method of any one of claims 16-26, wherein the subject exhibits decreased volume in tumor size following treatment.

30. The method of any one of claims 16-26, wherein the subject exhibits enhanced killing of cancer cells by the DNA damaging agent.

31. A combination therapy for treating or prevention of a CCR5+ cancer metastasis in a subject having cancer comprising administering an anti-CCR5 agent and DNA damaging chemotherapy.

32. The combination therapy of claim 31, wherein the cancer is a CCR5+ cancer selected from at least one of the following: leukemia cancer, lymphoma cancer, bone and connective tissue sarcoma, brain tumor cancer, breast cancer, adrenal cancer, pancreatic cancer, stomach cancer, colon cancer, prostate cancer, rectal cancer, gallbladder cancer, lung cancer, oral cancer, skin cancer, kidney cancer, and osteogenic sarcoma cancer.

33. The combination therapy of claim 32, wherein the cancer is breast cancer.

34. The combination therapy of claim 33, wherein the cancer is triple negative breast cancer (TNBC).

35. The combination therapy of claim 31, wherein the anti-CCR5 agent comprises antibodies or fragments thereof, non-antibody proteins or fragments thereof, and small molecule agents.

36. The combination therapy of claim 35, wherein the anti-CCR5 agent comprises an antibody, or a fragment thereof.

37. The combination therapy of claim 36, wherein the anti-CCR5 agent is leronlimab, or a fragment thereof.

38. The combination therapy of claim 31, wherein the DNA damaging agent comprises, wherein the DNA damaging chemotherapy is selected from one of doxorubicin, carboplatin, daunorubicin, epirubicin, idarubicin, mitoxantrone, and ametantrone, and any derivatives thereof.

39. The combination therapy of claim 38, wherein the DNA damaging agent comprises doxorubicin or carboplatin.

40. The combination therapy of claim 39, wherein the DNA damaging agent consists of doxorubicin.

41. The combination therapy of claim 39, wherein the DNA damaging agent consists of carboplatin.

42. The combination therapy of any one of claims 31-41, wherein the subject exhibits a reduction in circulating tumor cells (CTC) or putative metastatic or cells in the peripheral blood following treatment.

43. The combination therapy of any one of claims 31-41, wherein the subject exhibits reduced CCR5 expression on cancer-associated cells after following treatment.

44. The combination therapy of any one of claims 31-41, wherein the subject exhibits decreased volume in tumor size following treatment.

45. The combination therapy of any one of claims 31-41, wherein the subjects exhibits enhanced killing of cancer cells by the DNA damaging agent.
